(19) United States
(12) Patent Application Publication
(10) Pub. No.: US 2023/0078736 A1
(43) Pub. Date: Mar. 16, 2023

(54) STEERABLE CATHETER

(71) Applicant: Lifesciences Innovation (Israel) Ltd., Caesarea (IL)

(72) Inventors: Haim Brauon, Beit Dagan (IL); Or Cohen, Modi'in (IL); Brian Patrick Murphy, Irvine, CA (US); Omar Fawzi Azanki, Rancho Santa Margarita, CA (US); Ernest William Heflin, Pleasanton, CA (US); Steven Eric Decker, Anaheim, CA (US); Timothy Allen Dalton, Garden Grove, CA (US); Ehud Aviv, Costa Mesa, CA (US); Lindsay Marie Hall, Murietta, GA (US); Murrad Mirza Kazalbash, Tustin, CA (US); Julia Akiko Roche, Costa Mesa, CA (US); Danny Barrientos Baldo, San Diego, CA (US)

(21) Appl. No.: 17/990,570

(22) Filed: Nov. 18, 2022

Related U.S. Application Data
(63) Continuation of application No. PCT/IB2021/054039, filed on May 12, 2021.
(60) Provisional application No. 63/067,805, filed on Aug. 19, 2020.
(60) Provisional application No. 63/027,329, filed on May 19, 2020.

(51) Int. Cl. A61M 25/01

(52) U.S. Cl. CPC A61M 25/0136; A61M 25/0138; A61B 2017/00243

(57) ABSTRACT

A delivery system includes a handle, a catheter shaft, and a steering mechanism. The catheter shaft extends from the handle to a flexible distal end portion. The steering mechanism is configured to steer the flexible distal end portion. The steering mechanism can include a steering element a control member, and one or more actuation elements. Actuating the control member can move the steering element to increase and/or decrease tension in one or more actuation elements.

### RELATED APPLICATIONS

The present application is a continuation of Patent Cooperation Treaty Application No. PCT/IB2021/054039, filed on May 12, 2021, which claims the benefit of U.S. Provisional Patent Application Ser. No. 63/027,329, filed on May 19, 2020, and U.S. Provisional Patent Application Ser. No. 63/067,805, filed on Aug. 19, 2020, which are incorporated herein by reference in their entireties for all purposes.

### BACKGROUND OF THE INVENTION

Endovascular delivery systems can be used in various procedures to deliver medical devices or instruments to a target location inside a patient's body that are not readily accessible by surgery or where access without surgery is desirable. The systems described herein can be used to deliver medical devices (stents, heart valve, grafts, clips, repair devices, valve treatment devices, etc.) to a location in a patient's body.

Access to a target location inside the patient's body can be achieved by inserting and guiding the delivery system through a pathway or lumen in the body, including, but not limited to, a blood vessel, an esophagus, a trachea, any portion of the gastrointestinal tract, a lymphatic vessel, to name a few. Catheters are known in the art and have been commonly used to reach target locations inside a patient's body.

In some procedures, a catheter is used to deliver a device for replacing, repairing and/or remodeling a native heart valve. The native heart valves (i.e., the aortic, pulmonary, tricuspid, and mitral valves) serve critical functions in assuring the forward flow of an adequate supply of blood through the cardiovascular system. These heart valves can be damaged, and thus rendered less effective, by congenital malformations, inflammatory processes, infectious conditions, or disease. Such damage to the valves can result in serious cardiovascular compromise or death. For many years the definitive treatment for such damaged valves was surgical repair or replacement of the valve during open heart surgery. However, open heart surgeries are highly invasive and are prone to many complications. More recently, transvascular techniques have been developed for introducing and implanting prosthetic devices in a manner that is much less invasive than open heart surgery. Transvascular techniques can be used for accessing the native mitral, aortic, tricuspid, and pulmonary valves.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The native tricuspid valve of the human heart connects the right atrium to the right ventricle. When operating properly, the leaflets of each heart valve function together as a one-way valve.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

Disclosed herein are delivery systems, steerable catheter, and related methods which can be used to deliver a medical device, tools, agents, or other therapy to a location within a body of a subject. In some implementations, the delivery systems or steerable catheter devices can be used to deliver a medical device through the vasculature, such as to a heart of the subject. For example, a flexible delivery catheter can be used to deploy valve repair and replacement devices at an implant site for the repair or replacement of poorly functioning native heart valves.

In some implementations, a delivery system for delivering a medical device, such as a replacement valve, a valve repair device, a valve remodeling device, etc., to a desired location is configured to be steered in a desired direction. The delivery system includes a handle, a catheter shaft, and a steering mechanism. The catheter shaft extends from a proximal end attached to the handle to a flexible distal end portion. The steering mechanism is attached to the handle and is configured to steer the flexible distal end portion.

In some implementations, the steering mechanism includes a steering element, a control member, and first and second actuation elements (e.g., control wires, pull wires, lines, sutures, wires, rods, etc.). The first and second actuation elements extend from proximal ends that are attached to the steering element to distal ends that are attached to the flexible distal end of the catheter shaft. Actuating the control member of the steering mechanism moves the steering element to increase tension in one of the first and second actuation elements and to release tension in the other of the first and second actuation elements.

In some implementations, the systems and/or devices herein can comprise one or more eccentrically positioned actuation elements (e.g., control wires, pull wires, lines, sutures, wires, rods, etc.) configured to cause a shaft to curve in a direction determined by a user, and/or to cause the shaft to straighten. The disclosed devices can further comprise a flexible, axially non-compressible sleeve (e.g., an actuation-element sleeve, a pull-wire sleeve, etc.) that extends co-axially over at least a portion of the actuation element or pull wire, with the sleeve free-floating within a lumen (e.g., an actuation-element lumen, a pull-wire lumen, etc.). The pull-wire sleeve is effective to reduce or eliminate disequilibrium caused by torqueing the shaft while in a contoured configuration and under the pulling force of the actuation element or pull wire, thereby enhancing the steerability and torqueability of the catheter device.

In some implementations, a steerable catheter device or delivery system comprises a shaft comprising a proximal portion, a distal portion, and a pull-wire lumen that extends at least partially through the proximal and distal portions. An actuation element, such as a pull wire, extends through the actuation-element lumen or pull-wire lumen and has a proximal end portion and a distal end portion, wherein the distal end portion of actuation element or pull wire is fixed to the distal portion of the shaft. An adjustment mechanism is operatively connected to the proximal end portion of the actuation element or pull wire and configured to increase and decrease tension in the actuation element or pull wire to adjust the curvature of the distal portion of the shaft. In some implementations, a telescopic tube extends over the actuation element or pull wire.

In some implementations, the distal portion of the shaft comprises a hypotube and the actuation elements or pull wires are connected to the hypotube.

In some implementations, a chain of links or rings (vertebrae) are present at the distal end portion of the catheter or catheter shaft to facilitate bending of the distal end portion of the catheter or catheter shaft.

In some implementations, the hypotube forms the backbone of the steerable catheter and is present over substantial length of the steerable catheter.

In some implementations, a steering mechanism (e.g., for a catheter, sheath, delivery system, etc.) comprises at least two threaded members (e.g., longitudinal members, elongate members, worm screws, steering screws, half screws, racks, etc.).

In some implementations, a steering mechanism (e.g., for a catheter, sheath, delivery system, etc.) comprises at least two racks.

In some implementations, the threaded portions, longitudinal portions, worm screws, half screws, racks, etc. are connected to actuation elements, such as pull wires, and apply tension to the actuation elements or pull wires. Tension applied to the first actuation element (e.g., the first pull wire) and/or the second actuation element (e.g., second pull wire) is effective to flex the distal portion away from the central axis of the shaft, wherein the direction of flexion is determined by the relative tensions in the two actuation elements or pull wires. In some implementations, more than two actuation elements or pull wires may be present to increase articulation of the catheter. In some implementations, the steerable catheter comprises at least two shafts.

Catheters or catheter shafts herein can include a flexible tube having a plurality of links. An actuation element (e.g., control wire, pull wire, etc.) can be connected to the plurality of links, rings, or vertebrae, such that applying tension to the actuation element or control wire causes the flexible tube to bend. Any of the steering mechanisms and/or control elements herein can be used with any of the catheter or catheter shaft designs herein to cause this bending and/or steering.

In some implementations, there is provided a delivery catheter of a delivery system includes a catheter shaft having a main lumen, a control-wire lumen (or other lumen), a plurality of links, and a control wire (or other actuation element). Each link is aligned with and connected to at least one adjacent link with a slot or cut formed between each pair of adjacent links.

In some implementations, a top portion of each link is narrower than a bottom portion of each link when the links are viewed from a side. Each link can include an orifice at the bottom of the link. Each link can include at least one slit. The actuation element, control wire, or pull wire can be connected to the plurality of links. Applying tension to the control wire causes the distal region (e.g., distal end portion) of the catheter shaft to bend. In some implementations, the links are formed by cutting a hypotube (or a portion thereof) into the desired shape of the links. In some implementations, the links are rings coupled together to form the catheter shaft or a portion thereof. This delivery catheter or delivery system can further include, integrate, combine with, and/or be used with any of the steering mechanisms and/or control elements herein as well as other features described elsewhere herein or shown in the various figures.

In some implementations, there is provided a delivery catheter of a delivery system includes a flexible tube, a first ring, a second ring, a single control wire, a plurality of links, and a coil sleeve. The flexible tube has a main lumen and a control wire lumen. The first and second rings are spaced apart in a distal region (e.g., distal end portion) of the flexible tube. The single control wire is in the control wire lumen and is connected to the first ring. The plurality of links are disposed in the distal region of the flexible tube between the first ring and the second ring. The links are cut from a single piece of material or a hypotube, such that each link is aligned with and connected to at least one adjacent link with a slot or cut formed between each pair of adjacent links, an orifice at the bottom of each link, and at least one slit in each link. The slit begins at the orifice and extends upward along at least a portion of the link. The coil sleeve is disposed in the control wire lumen around the control wire in a proximal region of the flexible tube. A portion of the control wire that extends from the second ring and to the first ring is not covered by the coil sleeve. This delivery catheter or delivery system can further include, integrate, combine with, and/or be used with any of the steering mechanisms and/or control elements herein as well as other features described elsewhere herein or shown in the various figures. Applying tension to the actuation element, control wire, or pull wire causes the distal region of the flexible tube to bend.

In some implementations, there is provided a catheter or catheter shaft includes a flexible tube, a plurality of links, a coiled tube, and a control wire. The flexible tube has a main lumen and a control wire lumen. Each of the links have a slit along a bottom region of the flexible tube. In a first configuration, the distal region of the flexible tube is straight, a top of each link is spaced apart from an adjacent link by a distance. In a second configuration, the distal region of the flexible tube is curved, the slits of each link are opened, and the distance between the top of each link has decreased such that the top of the distal region of the flexible tube defines a curve. This catheter or catheter shaft can further include, integrate, combine with, and/or be used with any of the steering mechanisms and/or control elements herein as well as other features described elsewhere herein or shown in the various figures.

In some implementations, there is provided a catheter or catheter shaft has a first flexible portion, a second flexible portion, and a control wire. The first flexible portion has a first stiffness. The second flexible portion has a second stiffness that is different from the first stiffness. The control wire extends along the first flexible portion and the second flexible portion to a distal end of the second flexible portion. The different stiffnesses can be configured based on cutting patterns in a hypotube of the catheter or catheter shaft, as well as by selecting various material properties. This catheter or catheter shaft can further include, integrate, combine with, and/or be used with any of the steering mechanisms and/or control elements herein as well as other features described elsewhere herein or shown in the various figures. Applying tension to the actuation element, control wire, or pull wire causes the first and second flexible portions of the flexible tube to bend to different radii.

In some implementations, there is provided a catheter or catheter shaft usable for delivering a device to a native valve of a patient's heart (e.g., a catheter or catheter shaft of a delivery system) comprises a flexible tube having a main lumen and a actuation element lumen or control wire lumen, and a plurality of links disposed in a distal region (e.g., distal end portion) of the flexible tube. In some implementations, each link is aligned with and connected to at least one adjacent link with a slot formed between each pair of adjacent links, wherein a top portion of each link is narrower than a bottom portion of each link when the links are viewed from a side, and wherein each link includes an orifice at the bottom of the link. In some implementations, each link includes at least one slit, wherein the slit begins at the orifice and extends upward along at least a portion of the link. The catheter or catheter shaft includes an actuation element in the actuation-element lumen (e.g., a control wire in the control wire lumen) that is connected to the plurality of links, such that applying tension to the actuation element or control wire causes the distal region of the flexible tube to bend. This catheter or catheter shaft can further include, integrate, combine with, and/or be used with any of the steering mechanisms and/or control elements herein as well as other features described elsewhere herein or shown in the various figures.

In some implementations, there is provided a catheter or catheter shaft usable for delivering a device to a native valve of a patient's heart (e.g., a catheter or catheter shaft of a delivery system) comprises a flexible tube having a main lumen and an actuation element lumen or a control wire lumen. The catheter or catheter shaft can also include a first ring in a distal region (e.g., distal end portion) of the flexible tube and a second ring in the distal region of the flexible tube that is spaced apart from the first ring. The first ring and the second ring can be pull rings, low-profile rings, and/or any other rings or vertebrae described herein. In some implementations, the catheter or catheter shaft includes a single actuation element in an actuation-element lumen (e.g., a single control wire in a control wire lumen) that is connected to the first ring.

In some implementations, a plurality of links, rings, or vertebrae can be disposed in the distal region of the flexible tube between the first ring and the second ring. In some implementations, a coil sleeve is at least partially disposed in the actuation element lumen or control wire lumen around the actuation element or control wire. The coil sleeve can be configured to extend proximally from the distal region of the flexible tube such that a portion of the single actuation element or single control wire that extends from the second ring and to the first ring is not covered by the coil sleeve.

The catheter or catheter shaft can be configured such that applying tension to the single actuation element (e.g., single control wire) causes the distal region of the flexible tube to bend. The catheter or catheter shaft can further include, integrate, combine with, and/or be used with any of the steering mechanisms and/or control elements herein as well as other features described elsewhere herein or shown in the various figures.

In some implementations, the catheter or catheter shaft also includes a coil sleeve disposed in the actuation element lumen or control wire lumen around the actuation element or control wire in a proximal region of the flexible tube. The coil sleeve can be configured to extend proximally from the distal region of the flexible tube such that a portion of the actuation element or control wire that extends from the second ring and to the first ring is not covered by the coil sleeve.

In some implementations, there is provided a delivery system for delivering a medical device to a desired location, the delivery system comprising: a handle, a catheter shaft extending from a proximal end attached to the handle to a flexible distal end portion, and a steering mechanism attached to the handle for steering the flexible distal end portion of the catheter shaft.

In some implementations, the steering mechanism comprises a steering element, a control member, a first actuation element extending from a proximal end attached to the steering element to a distal end attached to the flexible distal end portion of the catheter shaft, and a second actuation element extending from a proximal end attached to the steering element to a distal end attached to the flexible distal end portion of the catheter shaft.

In some implementations, the delivery system is configured such that actuation of the control member of the steering mechanism moves the steering element to increase tension in one of the first and second actuation elements and to release tension in and/or apply compression or pushing force to the other of the first and second actuation elements.

In some implementations, the delivery system in configured such that actuation of the control member moves the first and second actuation elements in opposite directions.

In some implementations, the delivery system in configured such that actuation of the control member facilitates bi-directional movement of the flexible distal end portion of the catheter shaft.

In some implementations, the first and second actuation elements are pull wires. In some implementations, the first and second actuation elements are round pull wires. In some implementations, the first and second actuation elements are flat pull wires.

In some implementations, the first and second actuation elements extend within first and second compression members, respectively.

In some implementations, the first and second compression members each extend from a proximal end that is attached to at least one of the handle, the catheter shaft, and the steering mechanism. In some implementations, the first and second compression members extend to distal ends thereof that are spaced apart or separated from the distal ends of the first and second actuation elements, respectively. In some implementations, the first and second compression members each have a length from the proximal end to the distal end that is less than a length of the first and second actuation members, respectively.

In some implementations, the first and second compression members are compression coils.

In some implementations, the control member engages the steering element via a threaded connection. In some implementations, the control member engages the steering element via a gear mechanism.

In some implementations, the first and second actuation elements are attached to the steering element by first and second grasping elements, respectively.

In some implementations, each of the first and second grasping elements comprise an adjusting member. In some implementations, the adjusting member is a worm screw and the first and second actuation elements comprise flat wires each including a plurality of slots for engaging the worm screws of the first and second grasping elements. In some implementations, the adjusting member is a textured wheel and the first and second actuation elements comprise flat wires. In some implementations, the adjusting member is a threaded knob and the first and second actuation elements comprise round wires each including a threaded portion for engaging the threaded knobs of the first and second grasping elements. In some implementations, the adjusting member is a toothed wheel and the first and second actuation elements comprise round wires each including a plurality of slots for engaging the toothed wheels of the first and second grasping elements.

In some implementations, the first and second actuation elements are attached to the steering element by a single grasping element. In some implementations, the single grasping element comprises first and second slots, first and second set screws arranged along the first and second slots, respectively, wherein proximal ends of the first and second actuation elements are inserted into the first and second slots and are retained by the first and second set screws, respectively. In some implementations, the first and second slots are arranged at either end of a single channel in the grasping element.

In some implementations, the grasping elements are integrally molded with the steering element. In some implementations, the grasping elements are retained in a pocket of the steering element. In some implementations, the pocket of the steering element comprises a retaining feature for securing the grasping elements within the pocket. In some implementations, the retaining feature is an undercut in the pocket.

In some implementations, the delivery system further comprises first and second supports for supporting the first and second actuating elements.

In some implementations, the supports are telescoping supports. In some implementations, the telescoping supports comprise: first and second tubes each extending between a distal end and a proximal end, wherein the proximal end of the first tube overlaps the distal end of the second tube. In some implementations, the first tube has a diameter that is less than a diameter of the second tube.

In some implementations, the delivery system further comprises: a stopper attached to at least one of the handle, the catheter shaft, and the steering mechanism; and a compression member surrounding at least one of the first and second actuation elements and extending from a proximal end that is attached to the stopper, wherein a service loop is formed in the compression member between the stopper and the catheter shaft. In some implementations, the stopper is moveable in at least one of a proximal direction and a distal direction to adjust a length of the service loop. In some implementations, the stopper is formed as a threaded body that is received in a threaded opening in a mounting portion.

In some implementations, the steering mechanism is housed within the handle.

In some implementations, the delivery system further comprises first and second steering mechanisms and first and second control members, wherein the first steering mechanism facilitates bending the flexible distal end portion in or along a first plane, and wherein the second steering mechanism facilitates bending the flexible distal end portion in or along a second plane.

In some implementations, the delivery system further comprises first and second handles, wherein the first steering mechanism is housed in the first handle and the second steering mechanism is housed in the second handle. In some implementations, the first handle comprises a first control member for engaging the first steering mechanism, and the second handle comprises a second control member for engaging the second steering mechanism.

In some implementations, the first actuation element extends distally from the steering element to the flexible distal end portion, and the second actuation element extends proximally from the steering element, around a pulley, and distally to the flexible distal end portion.

In some implementations, the delivery system further comprises a first stop fixedly attached to the first actuation element, and a second stop fixedly attached to the second actuation element between the steering element and the pulley. In some implementations, the first stop is spaced apart from the steering element by a first distance and the second stop is spaced apart from the steering element by a second distance.

In some implementations, actuation of the control member in a first direction moves the steering element distally to apply tension to the second actuation element and to release tension in the first actuation element, and actuation of the control member in a second direction moves the steering element distally to apply tension to the first actuation element and to release tension in the second actuation element.

In some implementations, the first actuation element and the second actuation element are formed from a single pull wire.

In some implementations, the steering element comprises a first steering portion attached to the first actuation element, and a second steering portion attached to the second actuation element.

In some implementations, the delivery system is configured such that actuation of the control member in a first direction moves the first steering portion proximally to apply tension to the first actuation element and the second steering portion distally to release tension to the second actuation element, and actuation of the control member in a second direction moves the first steering portion distally to release tension to the first actuation element and the second steering portion proximally to apply tension to the second actuation element.

In some implementations, a first threaded portion of the control member having threads in a first direction, a second threaded portion of the control member having threads in a second direction, a threaded portion of the first steering portion having threads in a first direction, and a threaded portion of the second steering portion having threads in a second direction. In some implementations, a diameter of the first and second threaded portions of the control member is the same. In some implementations, a diameter of one of the first and second threaded portions of the control member is greater than the diameter of the other of the first and second threaded portions.

In some implementations, the first and second threaded portions of the control member are arranged in series in an axial direction of the control member. In some implementations, the first and second threaded portions of the control member overlap each other in an axial direction of the control member. In some implementations, the first and threaded portions of the control member are formed from a bi-directional thread. In some implementations, the first threaded portion of the control member has a thread pitch that is different from a thread pitch of the second threaded portion of the control member.

In some implementations, the first and second threaded portions of the control member are formed on an inner diameter of the control member, and the threaded portions of the first and second steering portions are formed on outer diameters of the first and second steering portions.

In some implementations, the first and second threaded portions of the control member are formed on an outer diameter of the control member, and the threaded portions of the first and second steering portions are formed on inner diameters of the first and second steering portions.

In some implementations, the first threaded portion of the control member is formed on an inner diameter of the control member, the second threaded portion of the control member is formed on an outer diameter of the control member, the threaded portion of the first steering portion is formed on an outer diameter of the first steering portion, and the threaded portion of the second steering portion is formed on an inner diameter of the second steering portion.

In some implementations, the steering mechanism comprises a drive gear and a driven gear.

In some implementations, the delivery system further comprises a drive member extending between and engaging the drive gear and the driven gear, wherein the first and second actuation elements are attached to the drive member. In some implementations, the first and second actuation elements are attached to the driven gear.

In some implementations, the driven gear comprises a first rack attached to the first actuation element, and a second rack attached to the second actuation element.

In some implementations, one or more transmission gears engaging the drive gear and the driven gear. In some implementations, the one or more transmission gears form a reducing transmission between the drive gear and the driven gear.

In some implementations, the drive gear is driven by the control member.

In some implementations, the delivery system further comprises first and second drive gears driven by the control member. In some implementations, the driven gears are rack gears.

In some implementations, the control member comprises a first threaded portion having threads in a first direction and a second threaded portion having threads in a second direction. In some implementations, the first driven gear has threads in the first direction, and the second driven gear has threads in the second direction.

In some implementations, the first driven gear is attached to the first actuation element, and the second driven gear is attached to the second actuation element. In some implementations, the first and second driven gears are rack gears.

In some implementations, the first drive gear and first driven gear are arranged on an opposite side of the control device from the second drive gear and second driven gear.

In some implementations, the delivery system further comprises a first steering mechanism having a first control knob, and a second steering mechanism having a second control knob.

In some implementations, the drive gears and driven gears of the first steering mechanism are radially offset from the drive gears and driven gears of the second steering mechanism by about 90 degrees.

In some implementations, the delivery system is configured such that actuating the first control knob bends the flexible distal end portion of the catheter shaft in a first plane and actuating the second control knob bends the flexible distal end portion of the catheter shaft in a second plane that is orthogonal to the first plane.

In some implementations, the flexible distal end portion comprises a first bending portion and a second bending portion. In some implementations, bending characteristics of the first bending portion are different than bending characteristics of the second bending portion. In some implementations, the first bending portion has a stiffness that is different from a stiffness of the second bending portion. In some implementations, the first bending portion has a bend radius that is different from a bend radius of the second bending portion. In some implementations, the first bending portion has a length that is different from a length of the second bending portion. In some implementations, the first bending portion has a wall thickness that is different from a wall thickness of the second bending portion.

In some implementations, the flexible distal end portion comprises a hypotube extending from the catheter shaft to a distal end, a plurality of lateral cuts in the hypotube, and an anchor portion at the distal end of the hypotube for attaching the actuation elements of the steering mechanism.

In some implementations, the plurality of slits in the first bending portion have a spacing that is different than the plurality of slits in the second bending portion. In some implementations, the plurality of slits in the first bending portion have a width that is different than the plurality of slits in the second bending portion. In some implementations, the plurality of slits in the first bending portion have a radial position that is different than the plurality of slits in the second bending portion.

In some implementations, the anchor portion is a pull ring that is attached to the hypotube.

In some implementations, the anchor portion is a pull ring that is integrally formed with the hypotube.

In some implementations, the flexible distal end portion comprises a first ring arranged at the distal end of the first bending portion, a second ring arranged at the proximal end of the first bending portion and the distal end of the second bending portion, a third ring arranged at the proximal end of the second bending portion, a first group of actuation elements attached to the first ring, and a second group of actuation elements attached to the second ring.

In some implementations, the first and second groups of actuation elements comprises a plurality of actuation elements radially spaced apart around the catheter shaft.

In some implementations, the actuation elements in each of the first and second groups of actuation elements are spaced apart by about 180 degrees.

In some implementations, each of the first and second groups of the actuation elements comprise two actuation elements.

In some implementations, the actuation elements in each of the first and second groups of actuation elements are spaced apart by about 120 degrees.

In some implementations, each of the first and second groups of the actuation elements comprise three actuation elements.

In some implementations, actuation elements in the first group of actuation elements are radially offset by an offset amount from actuation elements in the second group of actuation elements. In some implementations, the offset amount is about 90 degrees. In some implementations, the offset amount is about 60 degrees.

In some implementations, the delivery system further comprises a first pair of compression members extending to the second ring and enclosing the first group of actuation elements, and a second pair of compression members extending to the third ring and enclosing the second group of actuation elements. In some implementations, the compression members comprise compression coils. In some implementations, the compression members are formed from nitinol.

In some implementations, the delivery system further comprises a hypotube extending through at least one of the first ring, the second, ring, and the third ring. In some implementations, the hypotube comprises a plurality of lateral cuts. In some implementations, the hypotube is integrally formed with the first ring, the second, ring, and the third ring.

In some implementations, the flexible distal end portion comprises: a plurality of links, each link comprising a ball joint and a socket joint, wherein the ball joints and socket joints of adjacent links are movably connected; and an anchor portion at the distal end of the flexible distal end portion for attaching the actuation elements of the steering mechanism. In some implementations, the actuation elements extend through openings in each link of the plurality of links. In some implementations, a plurality of springs is arranged between each of the plurality of links. In some implementations, the plurality of springs are arranged between adjacent actuation elements.

In some implementations, the delivery system further comprises a first group of actuation elements attached to the anchor portion and a second group of actuation elements attached to the anchor portion.

In some implementations, the first and second groups of actuation elements comprises a plurality of actuation elements radially spaced apart around the catheter shaft. In some implementations, the actuation elements in each of the first and second groups of actuation elements are spaced apart by about 180 degrees. In some implementations, each of the first and second groups of the actuation elements comprise two actuation elements.

In some implementations, the delivery system is configured such that actuation of the first group of actuation elements causes the flexible distal portion to bend in a first plane, and actuation of the second group of actuation elements causes the flexible distal portion to bend in a second plane.

In some implementations, the delivery system is configured such that actuation of the first group of actuation elements causes the flexible distal portion to rotate axially, and actuation of the second group of actuation elements causes the flexible distal portion to bend laterally.

In some implementations, the first group of actuation elements is actuated by a first steering mechanism and the second group of actuation elements is actuated by a second steering mechanism.

In some implementations, the first and second groups of actuation elements are actuated by a single steering mechanism.

In some implementations, the first and second grasping elements comprise first and second slots, a bearing member arranged between the first and second slots, and a set screw arranged along the second slot, wherein the proximal end of the actuation element is inserted through the first slot, wrapped around the bearing member, inserted into the second slot, and retained by the set screw.

In some implementations, the flexible distal end portion comprises a plurality of links, rings, or vertebrae, and wherein the first actuation element attaches to an intermediate ring, link, or vertebrae of the plurality of links, rings, or vertebrae and the second actuation element attaches to a distal ring, link, or vertebrae at or near a distal end of the plurality of links, rings, or vertebrae.

In some implementations, the delivery system is configured such that applying tension to the first actuation element bends the catheter shaft in a first direction and applying tension to the second actuation element bends the catheter shaft in a second direction, wherein the first direction and the second direction are in or along the same plane. In some implementations, the first direction and the second direction are opposite directions.

In some implementations, the plurality of links, rings, or vertebrae include protrusions and recesses configured to connect such that they inhibit rotation of the plurality of links, rings, or vertebrae except along a longitudinal axis of the delivery system.

In some implementations in a fully bent condition (or fully actuated condition), the distal end of the plurality of rings or vertebrae is arranged in or along the same plane as a proximal end of the plurality of rings or vertebrae.

In some implementations, there is provided a delivery system that comprises a catheter shaft, at least one actuation element (e.g., a pull wire, etc.), a distal end of said at least one actuation element or pull wire connected to a distal end portion (e.g., flexible distal end portion) of the catheter shaft, and a handle comprising a steering mechanism, said handle attached to the catheter shaft at a proximal end of the catheter shaft, a proximal end of the at least one actuation element or pull wire connected to the steering mechanism, wherein the steering mechanism is capable of applying a tensile force to the actuation element or pull wire to bend or curve the catheter shaft.

In some implementations, the delivery system includes a support structure to help prevent prolapse of the at least one actuation element or pull wire.

In some implementations, the support structure comprises a support tube surrounding the at least one actuation element or pull wire, the support tube being moveable inside a sleeve tube, said sleeve tube having an inside diameter greater than the outside diameter of the support tube, a proximal end of at least one of the sleeve tube and the support tube being connected to the steering mechanism.

In some implementations, an inside diameter of the support tube is substantially same as and greater than an outside diameter of the at least one actuation element or pull wire wherein the at least one actuation element or pull wire is circular.

In some implementations, the at least one actuation element or pull wire is a non-circular actuation element or pull wire and the inside diameter of the support tube is substantially same as and greater than a width of the non-circular actuation element or pull wire whereby the non-circular actuation element or pull wire moves freely inside the support tube.

In some implementations, when operating the steering mechanism a moving member of the steering mechanism advances or retracts, whereby the support tube and the sleeve tube move in a telescopic manner and provide encasing lumen to the actuation element or pull wire inside the support tube and the sleeve tube, thereby preventing prolapse of the actuation element or pull wire.

In some implementations, the support tube and the sleeve have flexibility that is substantially same as the flexibility of the catheter shaft.

In some implementations, the delivery system further comprises a plurality of low-profile rings, wherein the low-profile rings are integral portions of a hypotube forming a portion of the catheter shaft or are connected to the hypotube at the flexible distal end portion of the catheter shaft.

In some implementations, the hypotube comprises a first section and a second section adjacent to the first section, the first section being distal from the second section, a first low-profile ring being connected to the hypotube at a distal end of the first section, a second low-profile ring being connected to the hypotube at a proximal end of the first section, and a third low-profile ring being connected to the hypotube at a proximal end of the second section.

In some implementations, the delivery system further comprises a first set of actuation elements or pull wires and compression coils and a second set of actuation elements or pull wires and compression coils, each set comprising at least three actuation elements or pull wires, said at least three actuation elements or pull wires being spaced equally apart from each other, the first set of actuation elements or pull wires being connected to the first low-profile ring and the second set of actuation elements or pull wires being connected to the second low-profile ring, the first set of actuation elements or pull wires configured to articulate the first section of the hypotube and the second set of actuation elements or pull wires configured to articulate the second section of the hypotube.

In some implementations, the delivery system further comprises a first set of actuation elements or pull wires and compression coils and a second set of actuation elements or pull wires and compression coils, each set comprising two actuation elements or pull wires, said two actuation elements or pull wires being spaced 180 degrees apart from each other, the first set of actuation elements or pull wires being in a horizontal plane when being connected to the first low-profile ring and the second set of actuation elements or pull wires being in a vertical plane when connected to the second low-profile ring, the first set of actuation elements or pull wires configured to articulate the first section of the hypotube in a horizontal plane and the second set of actuation elements or pull wires configured to articulate the second section of the hypotube in a vertical plane.

In some implementations, each actuation element or pull wire in the first set of actuation elements or pull wires has the compression coil terminating proximal of the first low-profile ring and each actuation element or pull wire in the second set of actuation elements or pull wires has the compression coil terminating proximal of the second low-profile ring.

In some implementations, the actuation elements or pull wires from the first set of actuation elements or pull wires are connected to a first control element in a first handle and the actuation elements or pull wires from the second set of actuation elements or pull wires are connected to a second control element in a second handle.

In some implementations, the actuation elements or pull wires from the first set of actuation elements or pull wires are connected to a first control element and the actuation elements or pull wires from the second set of actuation elements or pull wires are connected to a second control element, the first control element and the second control element being located in a single handle.

In some implementations, each set of two actuation elements or pull wires comprises a first actuation element or pull wire and a second actuation element or pull wire, said first and second actuation elements or pull wires being connected to a steering mechanism, the steering mechanism comprising a pulley or a tube, and a knob, the two actuation elements or pull wires being two parts of one continuous wire going over the pulley or the tube, the first actuation element or pull wire comprising at least two plugs, said plugs being moved by a control element, wherein the control element moves in a forward direction when the knob is moved in a clockwise direction, and the control element moves in a backward direction when the knob is moved in a counterclockwise direction.

In some implementations, when the control element moves in the forward direction, the control element is configured to engage at least one plug at a distal end of the control element, pushing the plug forward and thereby pushing the first actuation element or pull wire in the forward direction and pulling the second actuation element or pull wire in the backward direction, and when the control element moves in the backward direction, the control element is configured to engage at least one plug at a proximal end of the control element, thereby pushing the second actuation element or pull wire in the forward direction and pulling the first actuation element or pull wire in the backward direction.

In some implementations, the control element comprises at least one clamp, the at least one clamp connecting the first actuation element or pull wire and the second actuation element or pull wire to the control element, and when the control element moves in the forward direction, the at least one clamp connected to the control element is configured to push the first actuation element or pull wire in the forward direction and pull the second actuation element or pull wire in the backward direction, and when the control element moves in the backward direction, the at least one clamp connected to the control element is configured to push the second actuation element or pull wire in the forward direction and pull the first actuation element or pull wire in the backward direction.

In some implementations, the at least one actuation element or pull wire is connected to a first control element of the steering mechanism in the handle, and a second actuation element or pull wire is connected to a second control element of a second steering mechanism in a second handle.

In some implementations, the at least one actuation element or pull wire is connected to a first control element of the steering mechanism and a second actuation element or pull wire is connected to a second control element of the steering mechanism, the first control element and the second control element being located in the handle.

In some implementations, the steering mechanism comprises a pulley or a tube, and a knob, the at least one actuation element or pull wire being one continuous wire going over the pulley or the tube.

In some implementations, the one continuous wire has at least two plugs or stops thereon, wherein the at least two plugs or stops are configured such that movement of a control element forward moves a portion of the one continuous wire in a first direction and movement of the control element backward moves the portion of the one continuous wire in a second direction opposite the first direction.

In some implementations, when the control element moves in the forward direction, the control element is configured to engage at least one plug at a distal end of the control element, pushing the plug forward and thereby pushing the portion of one continuous wire in the first direction, and when the control element moves in the backward direction, the control element is configured to engage at least one plug at a proximal end of the control element, thereby pulling the portion of the one continuous wire in the second direction.

In some implementations, the delivery system further comprises a knob, wherein rotation of the knob in a first rotational direction causes the control element to move forward, and rotation of the knob in a second rotational direction causes the control element to move backward.

In some implementations, the steering mechanism comprises a pulley or a tube, and a knob, wherein the at least one actuation element or pull wire is a first actuation element or pull wire and the delivery system further comprises a second actuation element or pull wire.

In some implementations, the first actuation element or pull wire and the second actuation element or pull wire are both coupled to a single control element such that movement of the control element in a first direction moves a first portion of the first actuation element or pull wire in the first direction and a second portion of the second actuation element or pull wire in a second direction, while movement of the control element in the second direction moves the first portion of the first actuation element or pull wire in the second direction and moves the second portion of the second actuation element or pull wire in the first direction.

In some implementations, the first actuation element or pull wire has a first plug or stop thereon and the second actuation element or pull wire has a second plug or stop thereon, wherein the first and second plugs or stops are configured such that movement of the control element in a first direction pushes on the first plug or stop to move a first portion of the first actuation element or pull wire in the first direction, while movement of the control element in the second direction pushes on the second plug or stop to move the second portion of the second actuation element or pull wire in the first direction.

In some implementations, the delivery system further comprises a knob, wherein rotation of the knob in a first rotational direction causes the control element to move in the first direction, and rotation of the knob in a second rotational direction causes the control element to move in the second direction.

In some implementations, the steering mechanism comprises at least one clamp, the at least one clamp connecting the at least one actuation element or pull wire to the control element, and when the control element moves in a first direction, the at least one clamp connected to the control element is configured to push the at least one actuation element or pull wire in the first direction and, when the control element moves in a second direction, the at least one clamp connected to the control element is configured to pull the at least one actuation element or pull wire in the second direction.

In some implementations, the at least one actuation element or pull wire comprises a first actuation element or pull wire and the delivery system further comprises a second actuation element or pull wire, the first and second actuation elements or pull wires being connected to the steering mechanism, wherein the steering mechanism comprises a first control element that pulls the first actuation element or pull wire and a second control element that releases tension and/or pushes on the second actuation element or pull wire providing bi-directional movement of the flexible distal end portion of the catheter shaft.

In some implementations, the first control element is a first threaded member, and wherein the second control element is a second threaded member. In some implementations, the first threaded member is a first half screw, and wherein the second threaded member is a second half screw.

In some implementations, the delivery system further comprises a gear mechanism that causes the first control element and/or the second control element to move.

In some implementations, the first control element is a first rack, wherein the second control element is a second rack, and wherein a pinion moves the first rack and the second rack in opposite directions as the pinion is actuated.

In some implementations, the first control element and the second control element are each at least one of a threaded member, rack, screw, and half screw, wherein the first control element includes threads in a first direction and the second control element includes threads in a second direction.

In some implementations, the first control element is a first portion of a toothed belt and the second control element is a second portion of the toothed belt, wherein the steering mechanism comprises at least one gear that interacts with the toothed belt to move the toothed belt.

In some implementations, the steering mechanism comprises at least two gears, the at least two gears providing a torque reduction using gears of at least two different diameters.

In some implementations, the delivery system further comprises a pocket provided at a proximal end of each control element to hold a actuation element clamp (e.g., a pull wire clamp, etc.), the pocket comprising an undercut in which the clamp is forced into place and held securely, thereby preventing the clamp from coming out or rotating during the operation of the steering mechanism.

In some implementations, the steering mechanism comprises at least one gear and a toothed belt.

In some implementations, the steering mechanism comprises at least two gears, the at least two gears providing a torque reduction using gears of at least two different diameters.

In some implementations, each actuation element or pull wire of the delivery system is connected to the steering mechanism at a wire-fix point, the wire-fix point comprising a one-part grasper.

In some implementations, the one-part grasper uses a pin to reduce load on the wire-fix point.

In some implementations, the delivery system further comprises at least two shafts, said at least two shafts comprising an outer shaft and an inner shaft, the outer shaft being configured to reach a location above a center of a mitral valve annulus or a tricuspid valve annulus, the outer shaft comprising at least two sequence bending sections, at least one of the two sequence bending sections being controlled by the steering mechanism for fine tuning movement of the outer shaft.

In some implementations, the inner shaft is configured to have at least a first motion and a second motion, the first motion being a clock-like rotational motion and the second motion being a steering motion for steering the inner shaft into the mitral valve or the tricuspid valve.

In some implementations, the inner shaft is capable of motions comprising clock-rotation motion, sweep motion and steering motion, and when the outer shaft is located above the center of the mitral valve annulus or the tricuspid valve annulus, the inner shaft is configured to advance to a point along the annulus, the advancing of the inner shaft being performed by using one or more motions of the inner shaft.

In some implementations, the catheter shaft is a flexible tube having a main lumen and an actuation element lumen (e.g., pull wire lumen, etc.).

In some implementations, the catheter shaft further comprises a plurality of links, rings, or vertebrae disposed in a distal region of the flexible tube.

In some implementations, each link of the plurality of links, rings, or vertebrae is aligned with and connected to at least one adjacent link with a slot formed between each pair of adjacent links, rings, or vertebrae, wherein a top portion of each link, ring, or vertebra is narrower than a bottom portion of each link, ring, or vertebra when the links, rings, or vertebrae are viewed from a side.

In some implementations, each link, ring, or vertebra includes an orifice at the bottom of the link, ring, or vertebra.

In some implementations, each link, ring, or vertebra includes at least one slit, wherein the slit begins at the orifice and extends upward along at least a portion of the link, ring, or vertebra.

In some implementations, the at least one actuation element or pull wire is at least partially positioned inside an actuation element lumen or pull wire lumen of the catheter shaft and is connected to the plurality of links, rings, or vertebrae, wherein applying tension to the actuation element or pull wire causes the distal end portion of the catheter shaft to bend.

In some implementations, the catheter shaft comprises a hypotube with cuts configured to facilitate bending of a distal end portion of the catheter shaft in a predictable way.

In some implementations, the catheter shaft comprises a plurality of links, rings, or vertebrae.

In some implementations, a first actuation element or pull wire attaches to a transition or intermediate link, ring, or vertebra arranged between a proximal end and a distal end of the plurality of links, rings, or vertebrae and a second actuation element or pull wire attaches to a distal link, ring, or vertebra arranged at the distal end of the plurality of links, rings, or vertebrae.

In some implementations, applying tension to the first actuation element or pull wire bends the catheter shaft in a first direction and applying tension to the second actuation element or pull wire bends the catheter shaft in a second direction.

In some implementations, the first direction and the second direction are in or along the same plane.

In some implementations, the first direction and the second direction are opposite directions.

In some implementations, in a fully bent condition, the distal end of the plurality of links, rings, or vertebrae is arranged in or along the same plane as a proximal end of the plurality of links, rings, or vertebrae.

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of embodiments of the present disclosure, a more particular description of the certain embodiments will be made by reference to various aspects of the appended drawings. It is appreciated that these drawings depict only typical embodiments of the present disclosure and are therefore not to be considered limiting of the scope of the disclosure. Moreover, while the figures can be drawn to scale for some embodiments, the figures are not necessarily drawn to scale for all embodiments. Embodiments and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 is another cutaway view of the human heart in a systolic phase showing mitral regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIG. 8 is a perspective view of an example of a hypotube that provides structure and control of a flex section at a distal portion of a steerable catheter or catheter shaft;

FIG. 9 is a schematic perspective cross-sectional view of the hypotube of FIG. 8;

FIG. 10 is a schematic front view of an example of a hypotube that forms a backbone of at least a part of a steerable catheter or catheter shaft;

FIG. 11 is an end view of the hypotube of FIG. 10;

FIG. 12 is a schematic cross-sectional view of the hypotube of FIG. 10;

FIG. 13 illustrates 3-dimensional steering of an example of a catheter including the example hypotube of FIGS. 10-11;

FIG. 14 is a front view of an example of a distal end portion of a steerable catheter or catheter shaft;

FIG. 15 is a side view of the distal end portion of the steerable catheter or catheter shaft of FIG. 14;

FIG. 16 is a cross-sectional view of the distal end portion of the steerable catheter or catheter shaft of FIG. 14 taken along plane 29 of FIG. 15;

FIG. 17 is an enlarged view of the area 30 of FIG. 16;

FIG. 18 is a perspective view of the distal end portion of the steerable catheter or catheter shaft of FIG. 14 with most of the springs and actuation elements removed;

FIG. 19 shows a side view of an example steering mechanism for an example delivery system;

FIG. 20 shows a side view of the example steering mechanism for the delivery system of FIG. 19;

FIG. 21 shows a side view of the example steering mechanism for the delivery system of FIG. 19;

FIG. 22 shows a side view of the example steering mechanism for the delivery system of FIG. 19;

FIG. 23 shows a side view of an example steering mechanism for an example delivery system;

FIG. 24 shows a front view of the example steering mechanism for the delivery system of FIG. 23;

FIG. 25 shows a side view of the example steering mechanism for the delivery system of FIG. 23;

FIG. 26 shows a front view of the example steering mechanism for the delivery system of FIG. 25;

FIG. 27 shows a perspective view of the example steering mechanism for the delivery system of FIG. 25;

FIG. 28 shows a side view of an example steering mechanism for an example delivery system;

FIG. 29 shows a front view of the example steering mechanism for the delivery system of FIG. 28;

FIG. 30 shows a side view of the example steering mechanism for the delivery system of FIG. 28;

FIG. 31 shows a front view of the example steering mechanism for the delivery system of FIG. 30;

FIG. 32 shows a perspective view of a rack from the example steering mechanism for the delivery system of FIG. 28;

FIG. 33 shows a perspective view of a worm gear the example steering mechanism for the delivery system of FIG. 28;

FIG. 34 shows a schematic cross-sectional view of an example steering mechanism for an example delivery system;

FIG. 35 shows a schematic front view of the example steering mechanism for the delivery system of FIG. 34;

FIG. 36 shows a schematic cross-sectional view of an example steering mechanism for a delivery system;

FIG. 37 shows a schematic back view of an example handle of an example steering mechanism for a delivery system;

FIG. 38 shows a schematic side view of the example handle of the example steering mechanism for the delivery system of FIG. 37;

FIG. 39 shows a cross-sectional view of the example handle of the example steering mechanism for the delivery system of FIG. 37;

FIG. 40 shows a schematic side view of an example handle of an example steering mechanism for an example delivery system;

FIG. 41 shows a schematic front view of the example handle of the example steering mechanism for the delivery system of FIG. 40;

FIG. 42 shows a perspective view of an example grasping element for retaining an actuation element for a delivery system;

FIG. 43 shows a cross-sectional view of the example grasping element for retaining an actuation element of FIG. 42;

FIG. 44 shows a partial front view of a steering mechanism for a delivery system including an example grasping element for retaining an actuation element;

FIG. 45 shows a cross-sectional view of the steering mechanism including the example grasping element for retaining an actuation element of FIG. 44 taken along the line A-A;

FIG. 46 shows a cross-sectional view of the steering mechanism of FIG. 44 taken along the line A-A without the example grasping element for retaining an actuation element;

FIG. 47 shows a perspective view of the grasping element for retaining an actuation element of FIG. 44;

FIG. 48 shows a schematic cross-sectional view of an example telescoping tube structure for actuation elements of a steering mechanism for a delivery system;

FIG. 49 illustrates 3-dimensional steering of an example of a catheter or catheter shaft of a delivery system including an example steering mechanism;

FIG. 50 shows a front view of an example control handle including a steering mechanism for steering of steerable catheter or catheter shaft;

FIG. 51 shows a perspective view of an example steering mechanism of a delivery system;

FIG. 52 shows a front view of the example steering mechanism of FIG. 51;

FIG. 53 shows a cross-sectional perspective view of the example steering mechanism of FIG. 51;

FIG. 54 shows a cross-sectional front view of the example steering mechanism of FIG. 51;

FIGS. 55-56 show a schematic front view of an example steering mechanism for a delivery system;

FIG. 57 shows a perspective view of an example grasping element for retaining an actuation element for a delivery system;

FIG. 58 shows a cross-sectional view of the example grasping element for retaining an actuation element of FIG. 57;

FIG. 59 shows a perspective view of an example grasping element for retaining an actuation element for a delivery system with the actuation element in a retracted position;

FIG. 60 shows a cross-sectional view of the example grasping element for retaining an actuation element of FIG. 59 with the actuation element in a retracted position;

FIG. 61 shows a schematic view of an example grasping element for retaining an actuation element for a delivery system;

FIG. 62 shows a schematic view of an example grasping element for retaining an actuation element for a delivery system;

FIG. 63 shows a schematic view of an example grasping element for retaining an actuation element for a delivery system;

FIG. 64 shows a schematic view of an example steering mechanism for a delivery system;

FIG. 65 shows a schematic view of an example steering mechanism for a delivery system;

FIGS. 66-67 show perspective views of an example control handle with an example steering mechanism for a delivery system;

FIG. 68 shows a perspective cross-sectional view of the example control handle with the example steering mechanism of FIG. 66;

FIG. 69 shows a front cross-sectional view of the example control handle with the example steering mechanism of FIG. 66;

FIG. 70 shows a perspective view of the example steering mechanism of the example control handle of FIG. 66;

FIG. 71 shows a front view of the example steering mechanism of FIG. 70;

FIG. 72 shows a top view of the example steering mechanism of FIG. 70;

FIG. 73 shows a side view of the example steering mechanism of FIG. 70;

FIG. 74 is a perspective view of an example of distal end portion of a steerable catheter in a curved or bent condition;

FIG. 75 is a side view of the distal end portion of the steerable catheter or catheter shaft of FIG. 74;

FIG. 76 is a bottom view of the distal end portion of the steerable catheter or catheter shaft of FIG. 74;

FIG. 77 is a front view of the distal end portion of the steerable catheter or catheter shaft of FIG. 74;

FIG. 78 is a rear view of the distal end portion of the steerable catheter or catheter shaft of FIG. 74;

FIG. 79 is a cross-sectional view of the distal end portion of the steerable catheter or catheter shaft of FIG. 74 taken along plane B-B of FIG. 78;

FIG. 80 is a cross-sectional view of the distal end portion of the steerable catheter or catheter shaft of FIG. 74 taken along plane C-C of FIG. 78;

FIG. 81 is a cross-sectional view of the distal end portion of the steerable catheter or catheter shaft of FIG. 74 taken along plane A-A of FIG. 78; and

FIG. 82 is a schematic view of the distal end portion of the steerable catheter or catheter shaft of FIG. 74 shown in a bent or curved condition during implantation of an implantable device.

### DETAILED DESCRIPTION

The following description refers to the accompanying drawings, which illustrate example implementations of the present disclosure. The drawings demonstrate several possible configurations of systems, devices, components, and methods that can be used for various aspects and features of the present disclosure. Other implementations having different structures and operation do not depart from the scope of the present disclosure. Specific examples provided herein are not intended to be limiting; for example, steering mechanisms described herein can also be adapted and used to steer other systems and devices not expressly described herein. As one example, various systems, devices, components, and methods are described herein that may relate to steerable delivery systems or steerable catheters. As a further example, Published PCT Patent Application No. WO2020/106705, which is incorporated by reference herein in its entirety, also describes various delivery systems or steerable catheters that can be used with the steering mechanisms, steering elements, and other features described herein.

Example implementations of the present disclosure and US Provisional Patent Application No. 63/027,329, filed May 19, 2020 (which is incorporated herein by reference in its entirety for all purposes) are directed to devices and methods for steering a flexible delivery system for a medical device, such as a catheter. These example delivery systems provide a wide range of motion for the positioning of a medical device and are versatile, reliable, and easy to use. For example, the delivery systems disclosed herein and/or disclosed in Provisional Patent Application No. 63/027,329 can be used to position and deploy an implantable medical device for use in the repair of a native heart valve. It should be noted that various implementations of native valve reparation devices and systems for delivery are disclosed herein and in Provisional Patent Application No. 63/027,329, and any combination of these options can be made unless specifically excluded. In other words, individual components of the devices and systems disclosed herein and/or disclosed in Provisional Patent Application No. 63/027,329 can be combined unless mutually exclusive or otherwise physically impossible.

As described herein, when one or more components are described as being connected, joined, affixed, coupled, attached, or otherwise interconnected, such interconnection may be direct as between the components or may be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of).

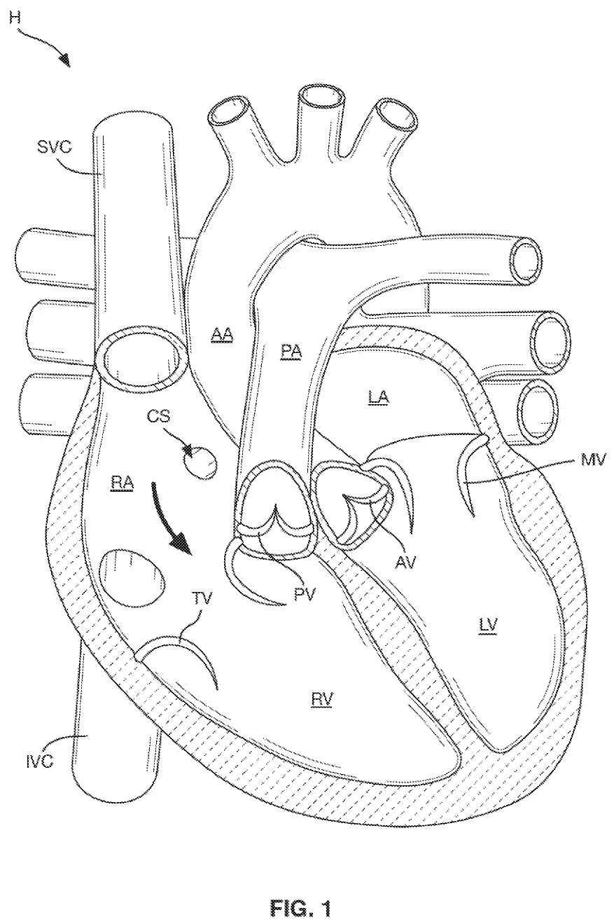

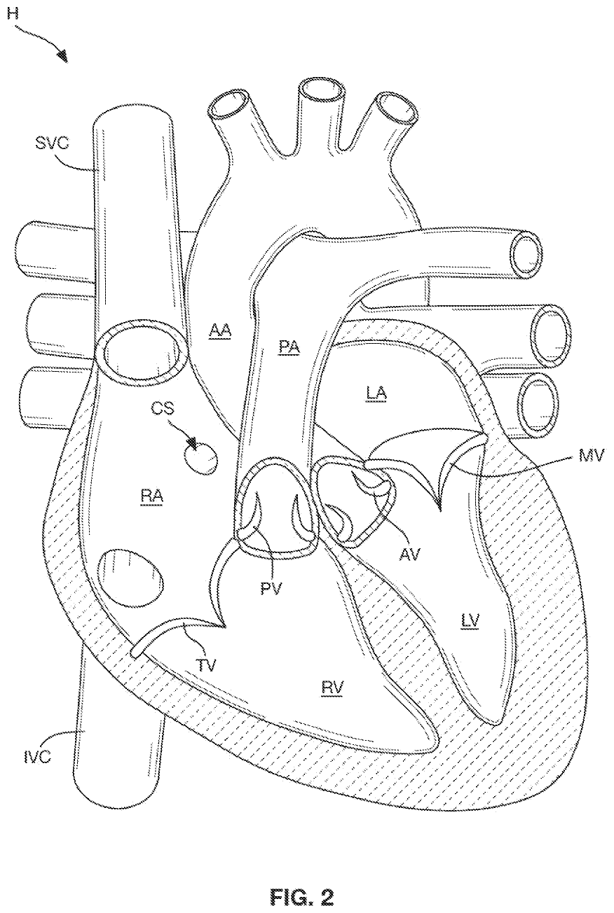

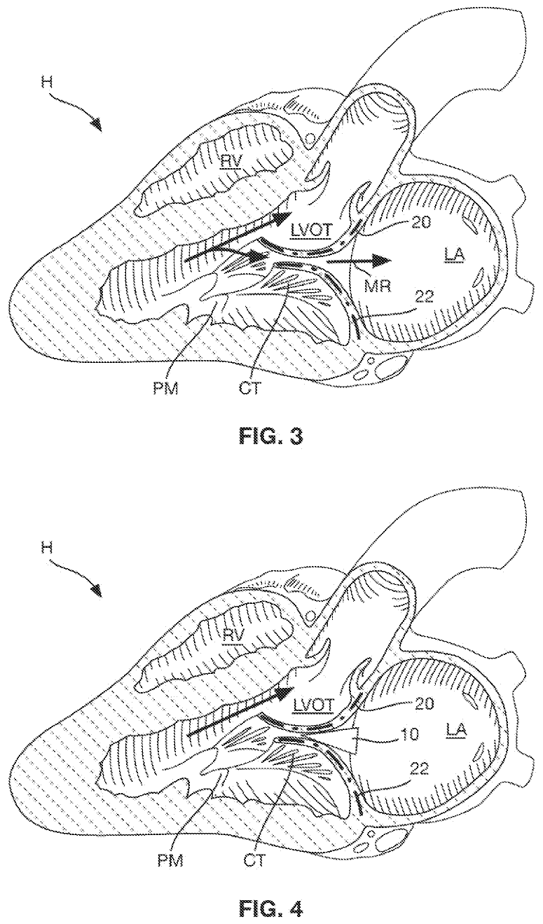

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV, respectively; i.e., the atrioventricular valves. The aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-7) extending inward across the respective orifices that come together or “coapt” in the flow stream to form the one-way, fluid-occluding surfaces. The devices and systems disclosed herein can be used to replace, repair, remodel, etc. the mitral valve MV, the tricuspid valve TV, the aortic valve AV, and/or the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV back into the left atrium LA and blood is collected in the left atrium from the pulmonary vein.

Referring now to FIGS. 1-7, the mitral valve MV includes two leaflets, the anterior leaflet 20 and the posterior leaflet 22. The mitral valve MV also includes an annulus 24, which is a variably dense fibrous ring of tissues that encircles the leaflets 20, 22. Referring to FIG. 3, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV to prevent the mitral valve MV from being reverted. The mitral valve MV opens and closes in response to relative pressure changes in the left atrium LA and the left ventricle LV. The papillary muscles PM do not open or close the mitral valve MV. Rather, the papillary muscles PM support or brace the leaflets 20, 22 against the high pressure necessary to circulate blood throughout the body. Together the papillary muscles PM and the chordae tendineae CT are known as the subvalvular apparatus, which functions to keep the mitral valve MV from prolapsing into the left atrium LA when the mitral valve closes.

As seen from a Left Ventricular Outflow Tract (LVOT) view shown in FIG. 4, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus. As a result, the leaflets 20, 22 form a space having a generally triangular shape 10 that is annotated in FIG. 4.

Various disease processes can impair proper function of one or more of the native valves of the heart H. These disease processes include degenerative processes (e.g., Barlow's Disease, fibroelastic deficiency), inflammatory processes (e.g., Rheumatic Heart Disease), and infectious processes (e.g., endocarditis). In addition, damage to the left ventricle LV or the right ventricle RV from prior heart attacks (i.e., myocardial infarction secondary to coronary artery disease) or other heart diseases (e.g., cardiomyopathy) can distort a native valve's geometry, which can cause the native valve to dysfunction.

Generally, a native valve may malfunction in two different ways: (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. The second type of valve malfunction, valve regurgitation, occurs when the leaflets of the valve do not close completely thereby causing blood to leak back into the prior chamber (e.g., causing blood to leak from the left ventricle to the left atrium).

There are three mechanisms by which a native valve becomes regurgitant—or incompetent—which include Carpentier's type I, type II, and type III malfunctions. A Carpentier type I malfunction involves the dilation of the annulus such that normally functioning leaflets are distracted from each other and fail to form a tight seal—i.e., the leaflets do not coapt properly. Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction can be caused by rheumatic disease (Ma) or dilation of a ventricle (IIIb).

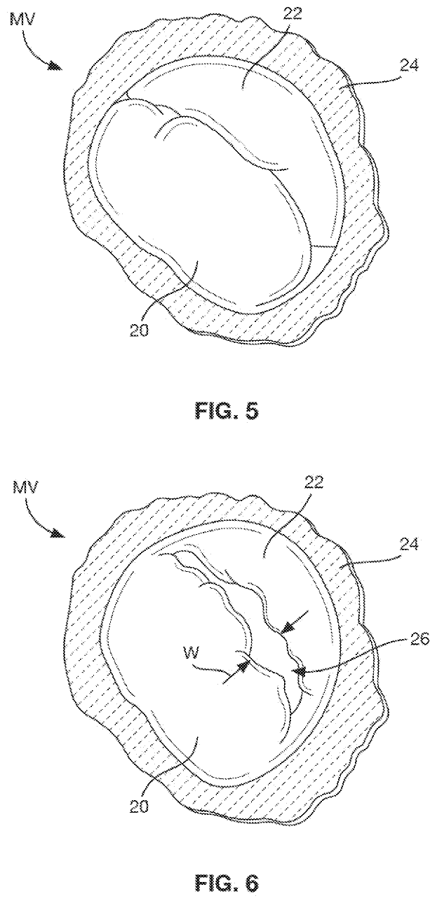

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22 that allows blood to flow back into the left atrium LA from the left ventricle LV during systole, as illustrated by the mitral regurgitation MR flow path shown in FIG. 6. As set forth above, there are several different ways that a leaflet (e.g. leaflets 20, 22 of mitral valve MV) may malfunction such that mitral regurgitation MR occurs.

Although stenosis or regurgitation can affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart is primarily responsible for circulating the flow of blood throughout the body, substantially higher pressures are experienced by the left side heart structures (i.e., the left atrium LA, the left ventricle LV, the mitral valve MV, and the aortic valve AV). Accordingly, malfunction of the mitral valve MV or the aortic valve AV is particularly problematic and often life threatening.

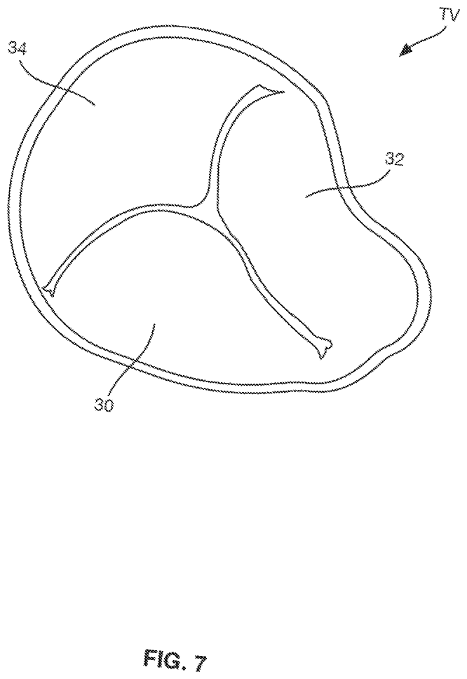

Malfunctioning native heart valves may either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, the most conventional treatments for a stenotic aortic valve or stenotic pulmonary valve are removal and replacement of the valve with a surgically implanted heart valve, or displacement of the valve with a transcatheter heart valve. The mitral valve MV and the tricuspid valve TV (FIG. 7) are more prone to deformation of annulus and/or leaflets, which, as described above, prevents the mitral valve MV or tricuspid valve TV from closing properly and allows for regurgitation or back flow of blood from the ventricle into the atrium (e.g., a deformed mitral valve MV may allow for mitral regurgitation MR or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation can occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV.

The devices and concepts provided herein can be used to replace any native valve, repair any native valve, remodel any native valve, as well as any component of a native valve. In one non-limiting example, a valve repair device can be used on native mitral leaflets 20, 22 or tricuspid leaflets 30, 32, 34.

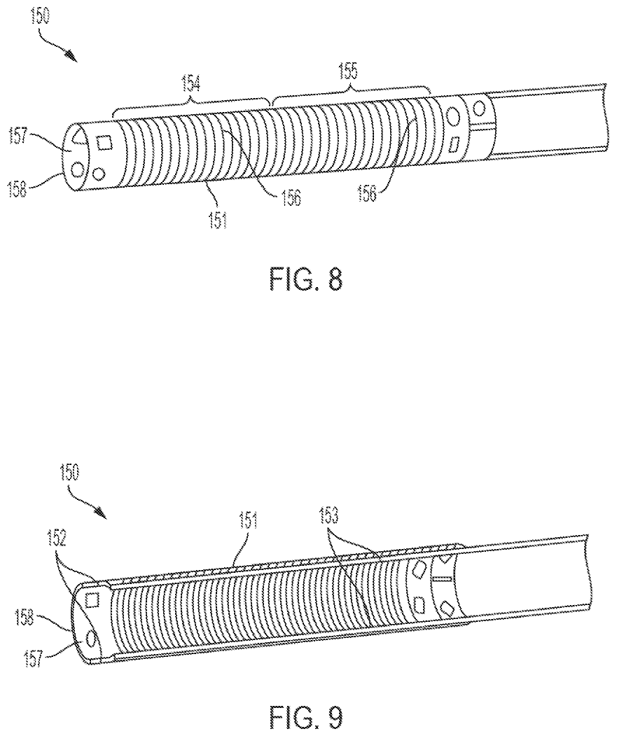

Referring now to FIGS. 8-9, an example steerable catheter 150 is shown that includes a hypotube 151 arranged at a distal portion of the catheter 150 to provide structure and control of the flex section at the distal portion of the catheter 150. The catheter 150 can include at least one shaft that includes the hypotube 151 and relies on the hypotube 151 as a backbone or spine of the entire shaft. The hypotube 151 can be co-centric with the rest of the catheter shaft. And can extend for the entire length or substantially the entire length of the catheter shaft. Optionally, the hypotube 151 can be located only at a distal portion or distal end portion of the shaft. A distal end 158 of the hypotube 151 can be aligned with or spaced apart from the distal portion or end of the catheter 150.

The hypotubes described herein, such as, for example, the hypotube 151, can be constructed using any suitable metal or alloy, such as, for example, stainless steel, nitinol, titanium, and the like. The hypotube 151 can also include an internal liner made from a material having substantially same properties as the material used to make lumens used in conjunction with other structures of the catheter, such as actuation elements (e.g., control wires, pull wires, lines, sutures, wires, rods, etc.) or compression members (e.g., compression coils). Additionally, a reflowed jacket or outer material can be provided over the entirety of the catheter shaft.

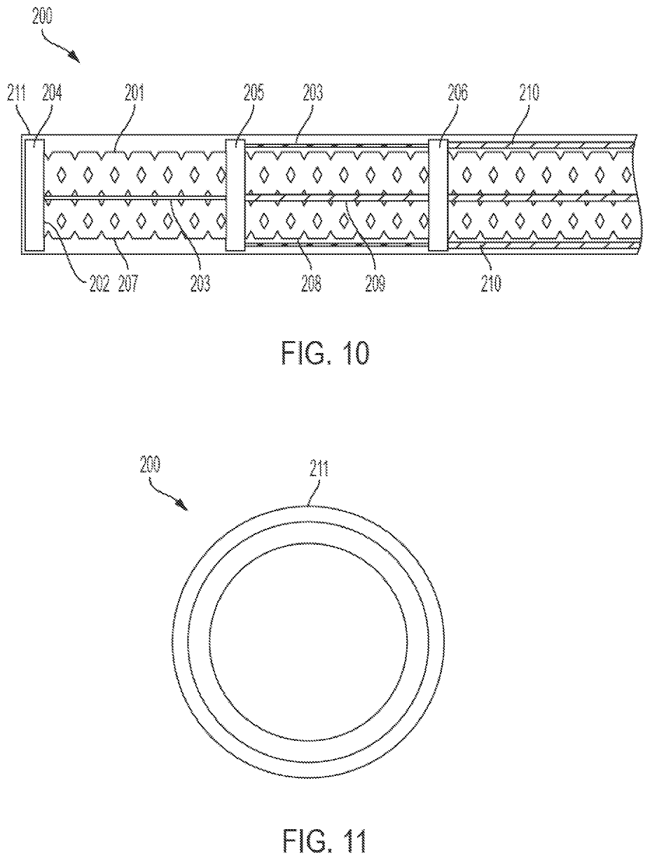

An operator of the catheter 150 can bend the distal portion of the catheter 150 by pulling or releasing actuation elements 153 that extend along the length of the hypotube 151. The actuation elements 153 can be pull wires having a circular or flat rectangular cross-sectional shape and can be secured to the hypotube 151 via a pull ring (see, e.g., FIG. 10) or via attachment locations 152. The actuation elements 153 can be directly attached to the attachment locations via welding, an adhesive, a mechanical fastener, or the like. For example, the actuation elements 153 can be laser welded to the attachment locations 152 of the hypotube 151.

A first or anchor portion 157 of the hypotube 151 proximal to the distal end 158 of the hypotube 151 can serve the same function as a pull-ring, that is, to attach to the actuation elements 153 so that tension applied to the actuation elements 153 is transmitted to the hypotube 151. Integrating a pull ring structure into the hypotube 151 prohibits misalignment of the pull ring and hypotube 151 that can occur when two separate components are joined together. The anchor portion 157 can be formed from an area of the hypotube 151 with a particular shape and/or increased strength, rigidity, and thickness to provide sufficient strength for receiving the actuation elements 153. To provide added stiffness and strength, the wall thickness of the anchor portion 157 of the hypotube 151 proximal to the distal end 158 of the hypotube 151 can be greater than the wall thickness of the remainder of the hypotube 151. For example, a thickness of a wall of an anchor portion 157 of the hypotube can be in the range of about 0.5 mm to about 2.5 mm. The anchor portion 157 of the hypotube 151 can also be wider than the remainder of the hypotube 151, the anchor portion 157 having a diameter in a range of about 5 mm to about 10 mm.

The hypotube 151 can be formed from a single section that bends substantially uniformly when actuated by the actuation elements 153. To facilitate bending, the hypotube 151 can include a plurality of relief cuts 156 that allow the hypotube 151 to flex and bend in one or more flexing directions with little or no axial compression under load. The relief cuts 156 can be formed in the hypotube 151 via laser cutting or any other suitable cuttings means to alter the bending characteristics of the hypotube 151. The relief cuts 156 can be formed in a variety of patterns, e.g., straight, spiral, staggered, zig-zag, etc. In some implementations, the repeating cuts 156 are aligned in a straight line along the axis of the shaft of the catheter. In some implementations, the repeating cuts 156 are staggered along the axis of the shaft of the catheter.

The hypotube 151 can include sections having different bending characteristics: that is, a first section 154 arranged near the distal end 158 of the hypotube 151 and a second section 155 proximal of the first section. That is, the first and second sections 154, 155 can differ in bend direction, bend rate, and bend radius when tension is applied to the actuation elements 153 to actuate the hypotube 151. The different bending characteristics of the first and second sections 154, 155 of the hypotube 151 can be provided in a wide variety of ways, such as, for example, by varying the thickness, stiffness, and material type of the first and second sections 154, 155.

The structure of the first and second sections 154, 155 can also be varied via the relief cuts 156 along the hypotube 151. The relief cuts 156 can be changed in their size, shape, and spacing along the length of the hypotube 151 and, in particular, between the first and second sections 154, 155 to provide different bending characteristics between the first and second sections 154, 155. For example, the bending direction can be altered between the first and second sections 154, 155. The spacing between consecutive cuts in the first section 154 can be greater than, the same as, or lesser than the spacing between consecutive cuts in the second section 155. In some implementations, the relief cuts 156 are formed such that links or link-like formations are formed in the hypotube 151.

Referring now to FIGS. 10-13, an example delivery system 200 is shown that includes a catheter or catheter shaft 211 and a hypotube 201 arranged within the catheter 211. The hypotube 201 extends along the catheter or catheter shaft 211 to a distal end 202 and can be caused to bend or flex via the actuation of a plurality of actuation elements 203 arranged around the circumference of the catheter or catheter shaft 211. The hypotube 201 can be divided longitudinally into sections that can be independently actuated by the actuation elements 203. In some implementations, the bending characteristics from one section of the hypotube 201 to another can be different.

The hypotube 201 includes first, second, and third ring sections 204, 205, 206 that provide support to the actuation elements 203 and also attachment locations for attaching the actuation elements 203 to the hypotube 201. The first ring section 204 is arranged at the distal end 202 of a first section 207 of the hypotube 201, the second ring section 205 is arranged at a mid-section between the first section 207 and a second section 208, and the third ring section 206 is arranged at a proximal end of the second section 208. While three ring sections 204, 205, 206 are shown in FIG. 2, the hypotube can include any suitable number of ring sections, such as, for example, 2, 3, 4, 5, or more ring sections that can correspond to a similar number of articulable sections of the hypotube 201.

The hypotube 201 can be formed from a tube of material or a sheet of material that is rolled and welded or otherwise joined along a seam. The tube or sheet of material can have a plurality of spaced apart cutouts arranged in a grid and each having a diamond shape (see FIG. 10) or any other suitable shape. For example, the cutouts can be formed as slits that extend around a majority of the circumference of the hypotube 201 to form a series of links having a rib cage like configuration. In some implementations, the links include a slot formed between each pair of adjacent links, a bottom orifice for each link, and at least one slit extending upward from the bottom orifice.

The ring sections 204, 205, 206 can have an outer diameter that is the same as the outer diameter of the hypotube 201. The outer diameter of one or more of the ring sections 204, 205, 206 can also be larger than the outer diameter of the hypotube 201 and smaller than an inside diameter of the catheter shaft—i.e., the diameter of the ring sections 204, 205, 206 can be larger than the diameter of the hypotube 201 yet be small enough to fit within the catheter or catheter shaft 211. The ring sections 204, 205, 206 can optionally be integrally formed in the hypotube 201 by cutting or otherwise forming the ring sections 204, 205, 206 in the material of the hypotube 201. Optionally, the ring sections 204, 205, 206 can be separate rings that are attached to the outer surface of the hypotube 201 via any suitable attachment means, such as, for example, welding, an adhesive, mechanical fastening, or the like.

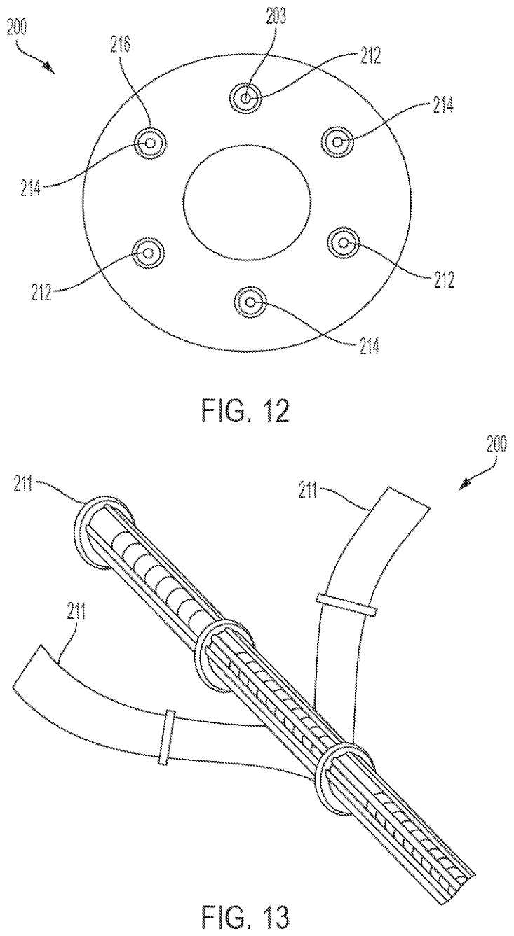

The actuation elements 203 are arranged into two groups: a first group 212 (FIG. 12) for articulating the first section 207 and a second group 214 for articulating the second section 208. The actuation elements 203 of the first group 212 extend through openings 216 in the second and third ring sections 205, 206 to attach to the first pull ring 204. The actuation elements 203 of the first group 212 extend through compression members 209 that terminate at the second pull ring 205. The actuation elements 203 of the second group 214 extend through openings 216 in the third ring section 206 to attach to the second pull ring 205. The actuation elements 203 of the second group 214 extend through compression members 210 that terminate at the third pull ring 206.

As can be seen in FIG. 12, the actuation elements 203 in each of the first and second groups 212, 214 are radially spaced apart from each other by about 120 degrees so that the three actuation elements 203 in each group are evenly spaced around the circumference of the hypotube 201. Optionally, one of the three actuation elements 203 in the first or second groups 212, 214 can be spaced apart about 135 degrees from the other two actuation elements 203. Additional actuation elements 203 can also be included such that the actuation elements 203 are radially spaced apart from other actuation elements 203 by about 90 degrees, or about 60 degrees, or about 30 degrees.

Applying tension to the actuation elements 203 causes the attached ring section 204, 205, 206 to move or tilt in the direction of the net tension force applied to the ring section 204, 205, 206. Consequently, the first or second section 207, 208 of the hypotube 201 that is immediately proximal of the articulated ring section 204, 205 is caused to flex or bend toward the applied force. Bending forces applied to one side of the hypotube 201 via one of the three actuation elements 203 in one of the first and second groups 212, 214 can be counteracted by forces applied to the other two actuation elements 203 in the first or second group 212, 204.

Providing three actuation elements 203 in each of the first and second groups 212, 214 enables each of the first and second sections 207, 208 to be articulated in any direction around the hypotube 201 by varying the amount of tension applied to each of the actuation elements 203. In particular, the direction of the bend in the hypotube 201 depends on the proportional distribution of tension forces in each of the three actuation elements 203 of the first or second groups 212, 214. That is, the relative proportion of tension applied to each of the three actuation elements 203—independent of the amount of tension applied—determines bend direction. The amount of tension applied to the actuation elements 203, however, is directly related to the magnitude of the bend in the hypotube 201; the greater the tension imbalance the greater the bend magnitude (i.e., the tighter or smaller the bend radius). Consequently, the catheter or catheter shaft 211 of the delivery system 200 can be articulated into a wide variety of positions, such as, for example, the range of positions shown in FIG. 13.

The actuation elements from each group 212, 214 can be connected to one or more steering elements of a steering mechanism, such as any example steering mechanisms disclosed herein, that is arranged in a handle (not shown) at a proximal end of the catheter or catheter shaft. For example, a first steering mechanism can be used to control the bending of the first section 207 and a second steering mechanism can be used to control the bending of the second section 208. Optionally, a single steering mechanism can control both of the first and second sections 207, 208. The steering mechanisms can be arranged in a single handle or in multiple handles such that each handle contains a single steering mechanism.

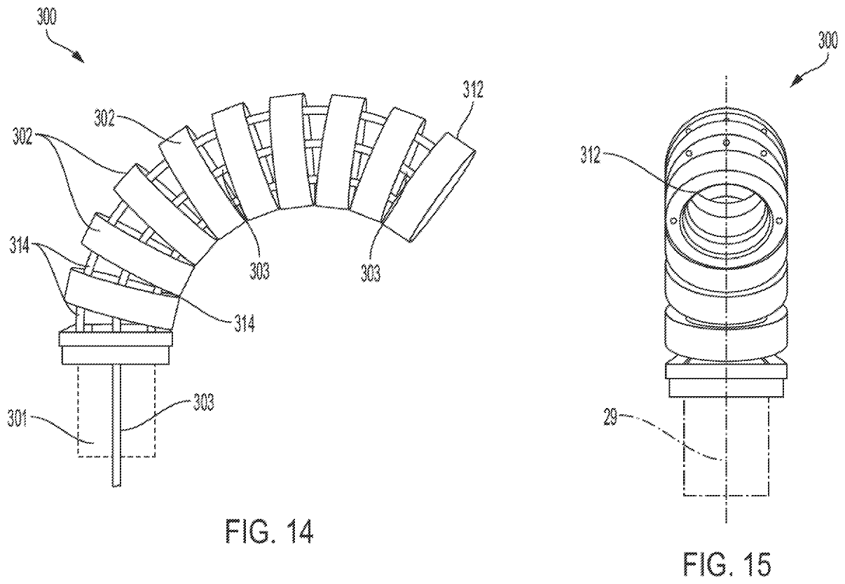

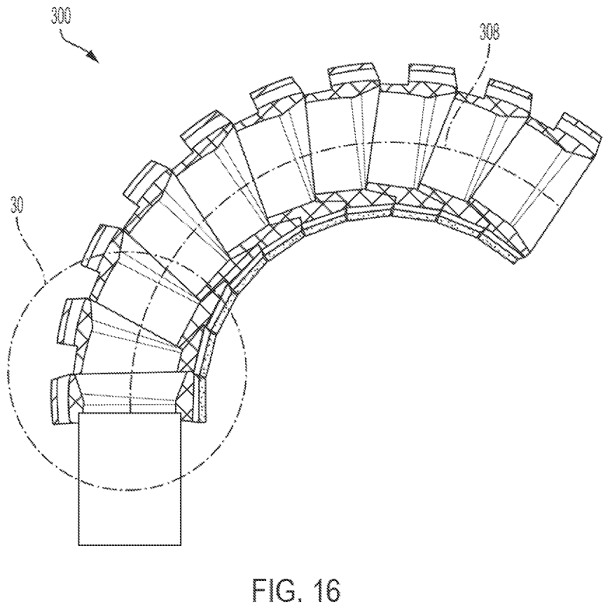

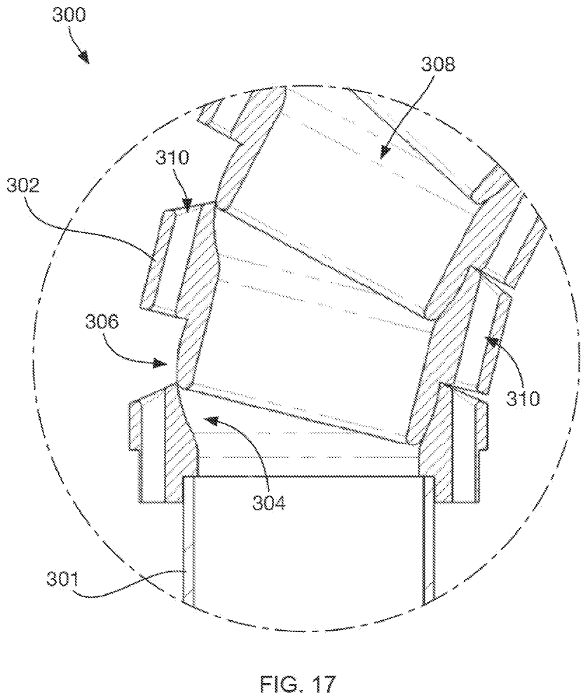

Referring now to FIGS. 14-18, an example delivery system 300 is shown. The delivery system 300 is formed from a plurality of links 302 arranged at a distal end portion of a catheter shaft 301. The links 302 operate similar to vertebrae of the human spine in that the links 302 include male and female connecting surfaces 304, 306 that provide a sliding joint between adjacent links 302. The connecting surfaces 304, 306 can be formed in a ball and socket joint configuration that allows the links 302 to pivot relative to one another. Each link 302 includes a central opening or lumen 308 and openings or lumen 310 for guiding and supporting actuation elements 303 that extend along the length of the delivery device 300 to a distal end 312. An optional hypotube (not shown) can also be provided that extends through the central openings 308 of the links 302.

The actuation elements 303 and actuation element openings 310 are radially spaced apart from each other by about 90 degrees to accommodate four actuation elements 303 extending along the length of the device 300 to the distal end 312. Applying tension to the actuation elements 303 causes the distal end 312 and the links 302 to move or tilt in the direction of the net tension force applied to the actuation elements 303. Consequently, the plurality of links 302 are caused to pivot relative to each other so that the device 300 flexes or bends toward the applied force. Bending forces applied to one side of the device 300 via one of the three actuation elements 303 can be counteracted by forces applied to the other actuation elements 303.

Providing four actuation elements 303 around the circumference of the device 300 enables the device 300 to be articulated in any direction around by varying the amount of tension applied to each of the actuation elements 303. In particular, the direction of the bend in the device 300 depends on the proportional distribution of tension forces in each of the four actuation elements 303. That is, the relative proportion of tension applied to each of the four actuation elements 303—independent of the amount of tension applied—determines bend direction. The amount of tension applied to the actuation elements 303, however, is directly related to the magnitude of the bend in the device 300; the greater the tension imbalance the greater the bend magnitude (i.e., the tighter or smaller the bend radius). Consequently, the end of the catheter or catheter shaft 301 of the delivery system 300 can be articulated into a wide variety of positions.

The actuation elements 303 can be connected to one or more steering elements of a steering mechanism, such as an example steering mechanisms disclosed herein, that is arranged in a handle (not shown) at a proximal end of the catheter or catheter shaft. For example, a first steering mechanism can be used to control the bending in a first bending plane of the device 300 so that the steering mechanism is connected to two actuation elements 303 that are spaced apart by 180 degrees around the device 300 and a second steering mechanism can be used to control the bending of the device 300 in a second bending plane that is orthogonal to the first bending plane. Optionally, a single steering mechanism can control bending in both the first and second bending planes. The steering mechanisms can be arranged in a single handle or in multiple handles such that each handle contains a single steering mechanism.

The actuation elements 303 can extend through compression members or coils (not shown) that extend from a handle or proximate the handle to a proximal portion of the most proximal link 302. The proximal side of the link 302 can include pockets or recesses for receiving a distal end of the compression member. Optionally, the compression member can run the entire length of the catheter shaft 301. In any of the catheter examples herein, each compression member can run through an individual lumen in a shaft of the catheter so that flexing of the shaft does not hinder independent movement of the compression member. In some implementations, the proximal face of a hypotube and/or links of a hypotube have bores and/or extensions to accept or abut against the compression members. The proximal face of the most proximal link 302 can also have bores and/or extensions to accept or abut against the compression members.

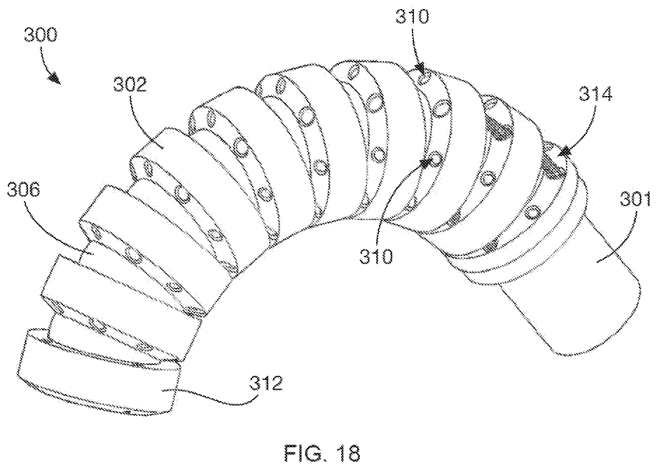

The device 300 can further include stiffening members arranged between the links 302. The stiffening members cause the device 300 to be biased in an extension direction so that the links 302 tend to straighten out after tension applied to the actuation elements 303 is relieved. The stiffening members can be formed in a tube shape from a shape-memory alloy, such as nitinol. As can be seen in FIG. 18, the stiffening members can be springs 314 that are biased in an expanding direction so that as the device 300 tends to straighten as tension applied to the actuation elements 303 is relieved. The springs 314 can be arranged between each pair of adjacent links 302 or can extend through multiple links 302. Four springs 314 can be arranged between each pair of adjacent links 302 so that the springs 314 are radially spaced apart by about 90 degrees and can be arranged between adjacent actuation elements 303 so that the springs 314 and actuation elements 303 alternate around the circumference of the device 300. Evenly spacing the springs 314 around the circumference of the links 302 evens out the forces applied to the links 302 and helps to maintain a symmetrical distance between adjacent rings 302.

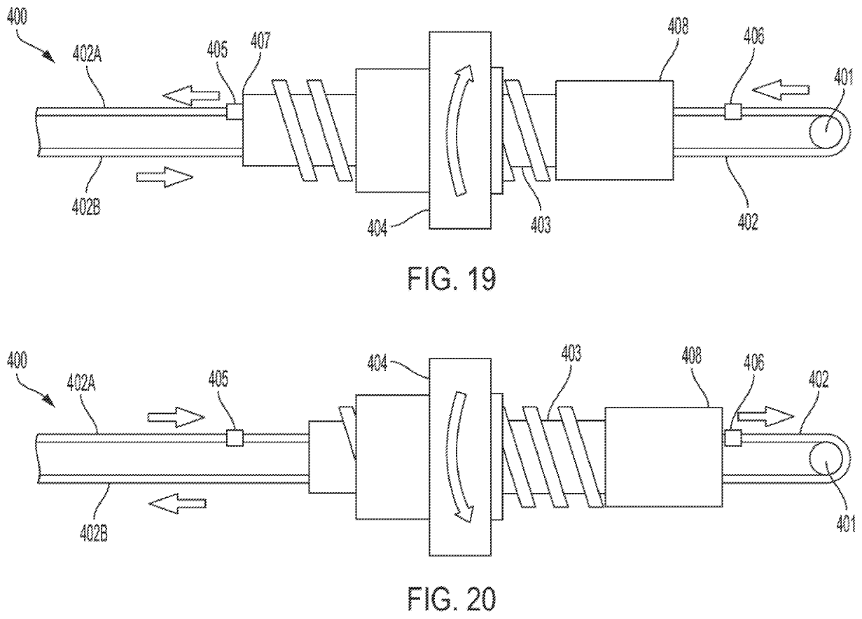

Referring now to FIGS. 19-22, an example steering mechanism 400 for a delivery system is shown. The steering mechanism 400 can be included inside of a handle of the delivery system and can be attached to proximal end of a catheter shaft. The steering mechanism 400 can include a pulley 401, one or more actuation elements 402, and a steering element 403. The pulley 401 can be formed as a wheel, tube, shaft, pin, or the like. The steering element 403 can be a threaded element (e.g., a screw, worm screw, steering screw, or the like), a translating member, a tube, a shaft, or the like. The pully can be made from a low friction material, such as, for example, polytetrafluoroethylene (PTFE).

The actuation element 402 is routed around the pulley 401 and through the steering element 403. Each end of the actuation element 402 attaches to a distal location in the delivery system, such as, for example, a pull ring, a low-profile ring, a hypotube, or the like. Each end of the actuation element 402 can extend through a compression member that extends for a portion of the actuation element 402 or for substantially the entire length of the actuation element 402. In some implementations, with two actuation elements, a first actuation element 402 can extend from a first attachment point in the delivery system, over the pulley 401, and to the steering element 403 and a second actuation element 402 can extend from a second attachment point in the delivery system to the steering element 403. The first and second actuation elements 402 can be attached to each other at the steering element 403 or can each be attached directly to the steering element 403. The actuation elements 402 can run parallel to each other through a lumen in a catheter shaft of the delivery system and can be partially or fully surrounded by compression members. The actuation elements 402 can be spaced apart by about 90 degrees, about 120 degrees, about 135 degrees, about 180 degrees, or by another amount around the catheter shaft, such as, for example, through a wall of the catheter shaft around a central delivery lumen.

The steering element 403 is actuated by a knob 404. The knob 404 can include internal threads on an inner surface of the knob 404 that directly interact with outer threads of the steering element 403 to cause the steering element 403 to translate forward and back. In some implementations, the internal threads of the knob 404 interact with a separate component—e.g., a separate threaded member, tube, gear, or the like—that interacts with the steering element 403. For example, the knob 404 can be coupled to a gear or gear assembly that causes the steering element 403 to translate forward and back so that the knob 404 does not require any internal threads. The knob 404 can be configured such that the steering element 403 moves in a forward direction when the knob 404 is rotated in a clockwise direction and the steering element 403 moves in a backward direction when the knob 404 is rotated in a counter-clockwise direction, or vice versa. Movement of the steering element 403 causes the one or more actuation elements 402 to move back and forth in a catheter shaft of the delivery system which can cause a distal region of the catheter to bend or straighten.

The steering mechanism 400 can also include one or more stops attached to the actuation element 402 such that movement of the steering element 403 does not cause the actuation element 402 to move until the steering element 403 engages one of the stops. Referring now to FIGS. 19 and 20, first and second stops 405, 406 are shown attached to the actuation element 402 on either side of the steering element 403. As the steering element 403 is moved in a first direction, the first stop 405 is engaged by a first end 407 of the steering element 403 so that a first portion 402A of the actuation element 402 is pushed by continued movement of the steering element 403 in the first direction and a second portion 402B of the actuation element 402 is pulled by continued movement of the steering element 403 in the first direction. As the steering element 403 is moved in a second direction, the second stop 406 is engaged by a second end 408 of the steering element 403 so that the first portion 402A of the actuation element 402 is pulled by continued movement of the steering element 403 in the second direction and the second portion 402B of the actuation element 402 is pushed by continued movement of the steering element 403 in the second direction. The first and second stops 405, 406 can be arranged on the actuation element 402 so that the first and second stops 405, 406 abut the first and second ends 407, 408 of the steering element 403 at all times, thereby minimizing slack or backlash between the movement of the steering element 403 and movement of the actuation element 402. Optionally, the first and second stops 405, 406 can be spaced apart such that movement of the steering element 403 in the opposite direction does not immediately reverse the movement of the actuation element 402.

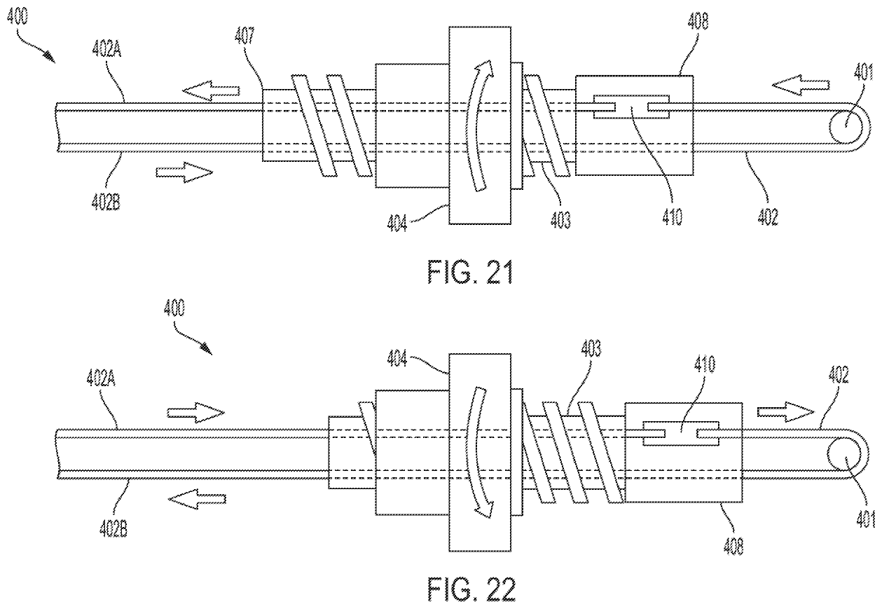

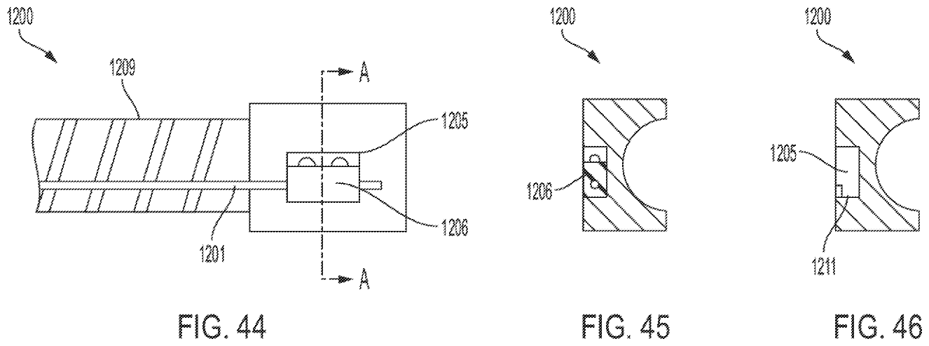

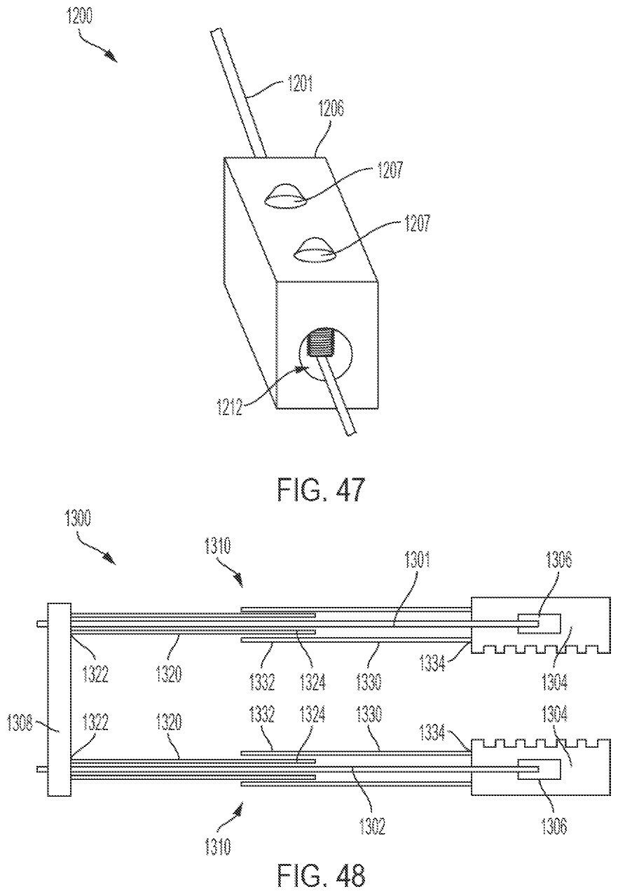

Referring now to FIGS. 21 and 22, the steering mechanism 400 can optionally include a grasping element 410 connected to the steering element 403. The grasping element 410 is connected to the ends of the first and second portions 402A, 402B of the actuation element 402. (An example of a clamp that can be used is shown in FIGS. 44-47.) As the steering element 403 is moved in the first direction the grasping element or clamp 410 engages the actuation element 402 so that the first portion 402A of the actuation element 402 is pushed by continued movement of the steering element 403 in the first direction and the second portion 402B of the actuation element 402 is pulled by continued movement of the steering element 403 in the first direction. As the steering element 403 is moved in the second direction the grasping element or clamp 410 engages the actuation element 402 so that the first portion 402A of the actuation element 402 is pulled by continued movement of the steering element 403 in the second direction and the second portion 402B of the actuation element 402 is pushed by continued movement of the steering element 403 in the second direction. Optionally, the first and second portions 402A, 402B of the actuation element 402 can be clamped by independent first and second clamps, respectively, that are each connected to the steering element 403.

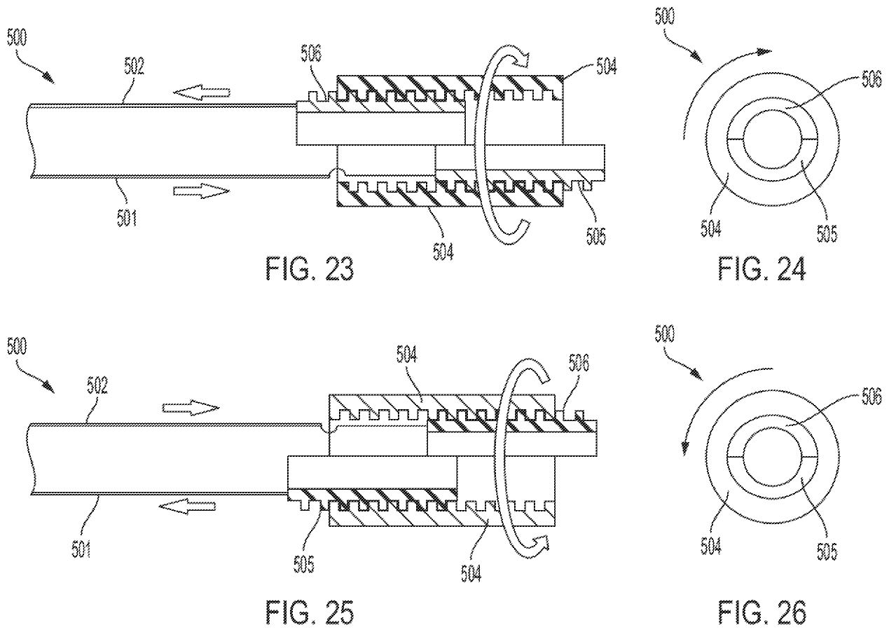

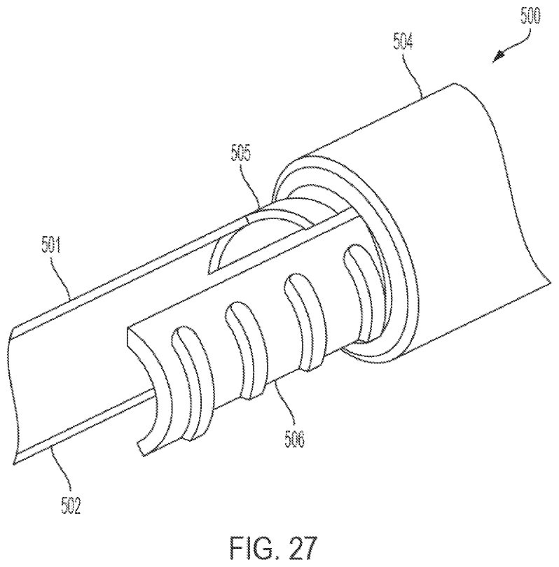

Referring now to FIGS. 23-27, an example steering mechanism 500 for a delivery system is shown. The steering mechanism 500 can be included inside of a handle of the delivery system and can be attached to proximal end of a catheter shaft. The steering mechanism 500 can include a knob 504, a first actuation element 501 connected to a first steering element 505, and a second actuation element 502 connected to a second steering element 506. The first steering element 505 has a right-hand thread and the second steering element 506 has a left-hand thread. The knob 504 includes an internal double thread for engaging the threads of the first and second steering elements 505, 506.

Referring now to FIGS. 23-24, when the knob 504 is rotated in the clockwise direction the first steering element 505 pulls or exerts tension on a first actuation element 501 (also known as a steering wire) and the second steering element 506 releases tension and/or pushes a second actuation element 502. Referring now to FIGS. 25-26, when the knob 504 is rotated in the counter-clockwise direction the first steering element 505 releases tension on and/or pushes the first actuation element 501 and the second steering element 506 pulls and/or exerts tension on the second actuation element 502. The simultaneous pulling and releasing/pushing of the actuation elements 501, 502 provides bi-directional movement of a distal end portion of the catheter. This movement is helpful particularly when the catheter has been held in a curved position for some time and has become set in the curved position, pushing and pulling on opposing actuation elements 501, 502 assists the operator in straightening the curved portion of the catheter shaft.

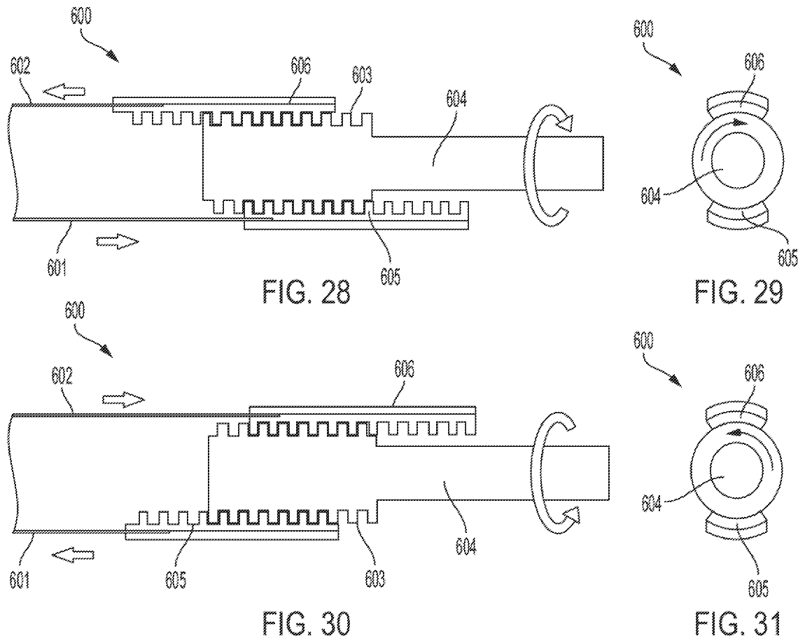

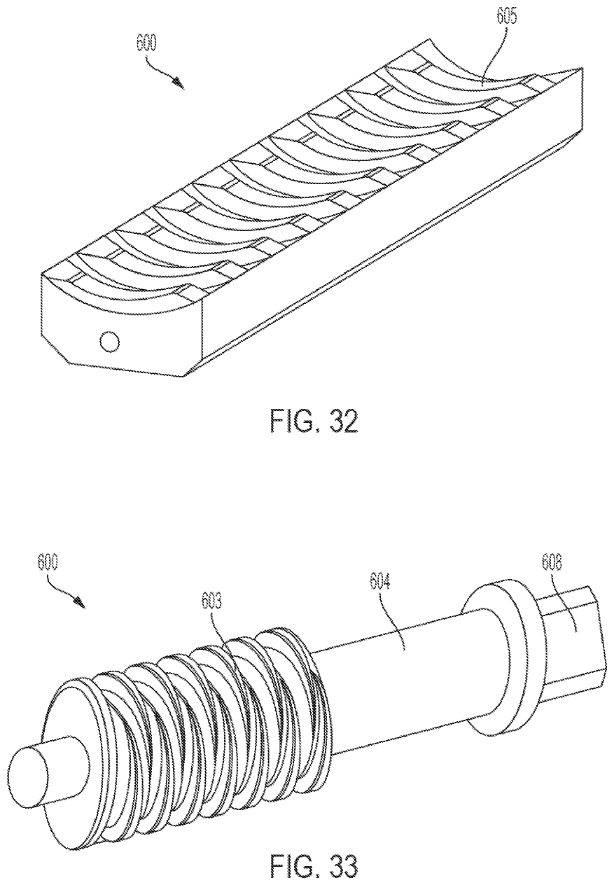

Referring now to FIGS. 28-33, an example steering mechanism 600 for a delivery system is shown. The steering mechanism 600 is capable of simultaneously pulling and releasing actuation elements 601, 602 and can be used with any delivery system disclosed herein. The steering mechanism 600 includes a double-threaded worm gear 604 and first and second racks 605, 606. A threaded portion 603 of the double-threaded worm gear 604 engages the first and second racks 605, 606 so that rotation of the worm gear causes the first and second racks 605, 606 to move proximally or distally. The first rack 605 has a right-hand thread and the second rack 606 has a left-hand thread. The first rack 605 is connected to the first actuation element 601 and the second rack 606 is connected to the second actuation element 602. The actuation elements 601, 602 are connected to the racks 605, 606 by any suitable means, such as with a grasping element disclosed herein.

When the double threaded worm gear 604 is rotated in the clockwise direction (FIGS. 28 and 29), the first rack 605 pulls the first actuation element 601 in a proximal direction and the second rack 606 moves in a distal direction to release the second actuation element 602. When the double threaded worm gear 604 is rotated in the counter-clockwise direction (FIGS. 30 and 31), the first rack 605 moves in a distal direction to release the first actuation element 601 and the second rack 606 pulls the second actuation element 602 in a proximal direction. The simultaneous pulling and releasing of the actuation elements 601, 602 provides bi-directional movement of a distal end portion of a catheter to which the actuation elements 601, 602 are attached. The double threaded worm gear 604 can be rotated via a knob 608 arranged at a proximal end portion of the worm gear 604.

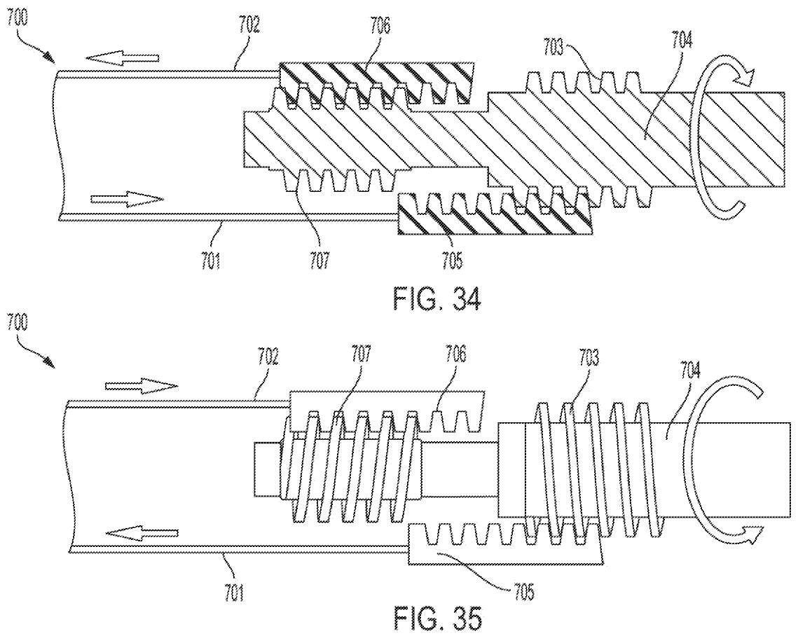

Referring now to FIGS. 34-35, an example steering mechanism 700 for a delivery system is shown. The steering mechanism 700 is capable of simultaneously pulling and releasing actuation elements 701, 702 and can be used with any delivery system disclosed herein. The steering mechanism 700 includes a bi-directional worm gear 704 having a proximal threaded portion 703 and a distal threaded portion 707. The threads of the proximal and distal threaded portions 703, 707 are opposite to each other, that is, the proximal threaded portion 703 has a right-hand thread when the distal threaded portion 707 has a left-hand thread, and vice versa. The diameter of the proximal and distal threaded portions 703, 707 can be the same or different. The proximal threaded portion 703 can have a greater diameter than the distal threaded portion 707, or vice versa. The steering mechanism 700 further includes a first rack 705 having threads that match those of the proximal threaded portion 703 and a second rack 706 having threads that match those of the distal threaded portion 707. The first rack 705 is connected to the first actuation element 701 and the second rack 706 is connected to the second actuation element 702. The actuation elements 701, 702 are connected to the racks 705, 706 by any suitable means, such as with a grasping element disclosed herein.

When the worm gear 704 is rotated in the clockwise direction (FIG. 34), the first rack 705 pulls the first actuation element 701 in a proximal direction and the second rack 706 moves in a distal direction to release the second actuation element 702. When the worm gear 704 is rotated in the counter-clockwise direction (FIG. 35), the first rack 705 moves in a distal direction to release the first actuation element 701 and the second rack 706 pulls the second actuation element 702 in a proximal direction. The simultaneous pulling and releasing of the actuation elements 701, 702 provides bi-directional movement of a distal end portion of a catheter to which the actuation elements 701, 702 are attached.

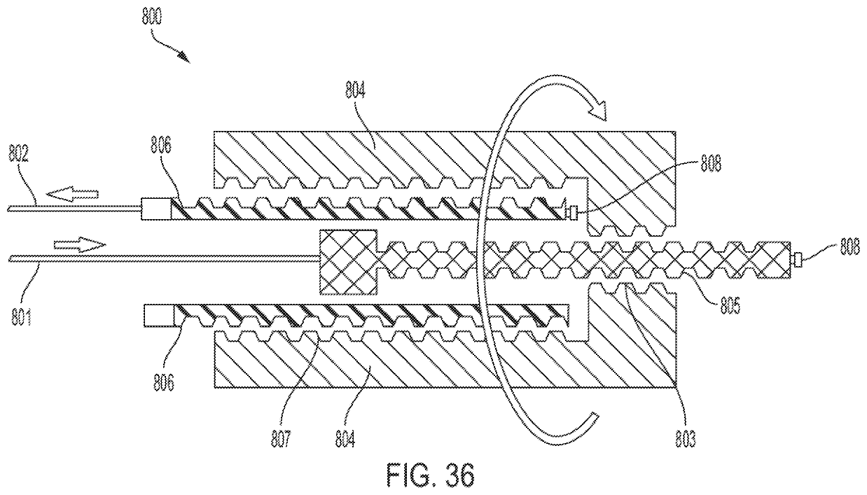

Referring now to FIG. 36, an example steering mechanism 800 for a delivery system is shown. The steering mechanism 800 is capable of simultaneously pulling and releasing actuation elements 801, 802 and can be used with any delivery system disclosed herein. The steering mechanism 800 includes a control member or knob 804 having internal threaded portions: a proximal threaded portion 803 and a distal threaded portion 807. The threads of the proximal and distal threaded portions 803, 807 are opposite to each other, that is, the proximal threaded portion 803 has a right-hand thread when the distal threaded portion 807 has a left-hand thread, and vice versa. The diameter of the proximal and distal threaded portions 803, 807 can be the same or different. The proximal threaded portion 803 can have a smaller diameter than the distal threaded portion 807, as shown in FIG. 36, or vice versa. The steering mechanism 800 further includes a first steering element 805 having threads that match those of the proximal threaded portion 803 and a second steering element 806 having threads that match those of the distal threaded portion 807. The first steering element 805 is connected to the first actuation element 801 and the second steering element 806 is connected to the second actuation element 802. The actuation elements 801, 802 are connected to the steering elements 805, 806 by any suitable means, such as with a grasping element disclosed herein.

When the control member 804 is rotated in the clockwise direction (FIG. 34), the first steering element 805 pulls the first actuation element 801 in a proximal direction and the second steering element 806 moves in a distal direction to release the second actuation element 802. When the control member 804 is rotated in the counter-clockwise direction, the first steering element 805 moves in a distal direction to release the first actuation element 801 and the second steering element 806 pulls the second actuation element 802 in a proximal direction. The simultaneous pulling and releasing of the actuation elements 801, 802 provides bi-directional movement of a distal end portion of a catheter to which the actuation elements 801, 802 are attached.

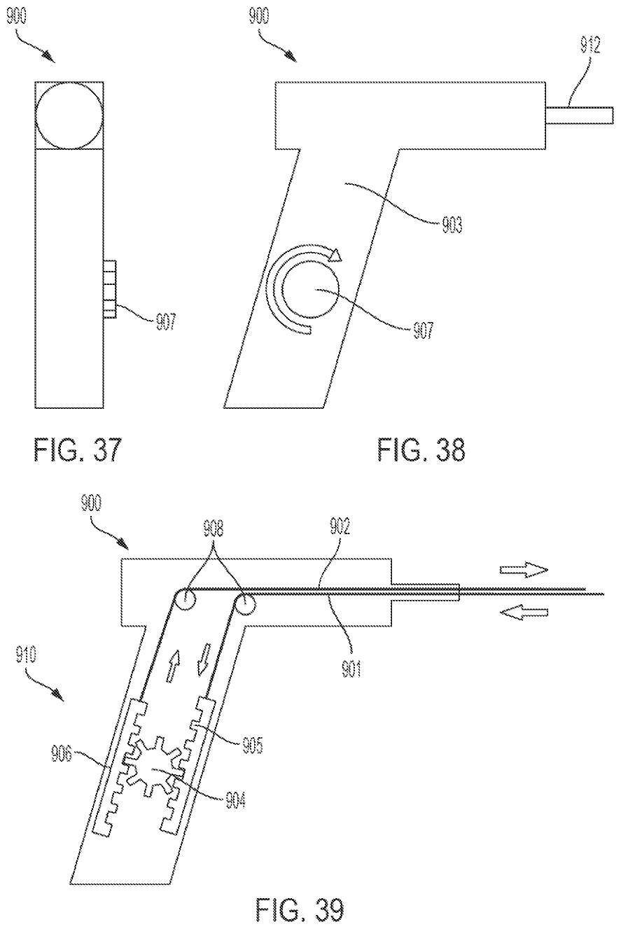

Referring now to FIGS. 37-39, an example control handle 900 including a steering mechanism 910 for a delivery system is shown. The handle 900 is connected to a proximal end of a catheter shaft 912. A grip portion 903 of the control handle 900 is configured for an operator to grasp the control handle 900 and to operate a control member 907 that actuates the steering mechanism 910. The steering mechanism 910 is capable of simultaneously pulling and releasing actuation elements 901, 902 that extend through the catheter shaft 912. The steering mechanism 910 includes a pinion gear 904 that engages toothed portions of first and second steering elements 905, 906. The first steering element 905 is connected to the first actuation element 901 and the second steering element 906 is connected to the second actuation element 902. The actuation elements 901, 902 are connected to the steering elements 905, 906 by any suitable means, such as with a grasping element disclosed herein. The actuation elements 901, 902 extend upward through the grip portion 903 of the control handle 900 and are redirected toward the catheter shaft 912 via pulleys 908. The actuation elements 901, 902 can be redirected by any suitable means, such as, for example, by eyelets, wheels, tubes, internal bearing surfaces handle, or the like.

When the control member 907 is rotated in the clockwise direction (FIG. 38), the pinion gear 904 similarly rotates to cause the first steering element 905 to pull the first actuation element 901 in a proximal direction and the second steering element 906 to move in a distal direction to release the second actuation element 902. When the control member 907 and pinion gear 904 are rotated in the counter-clockwise direction, the first steering element 905 moves in a distal direction to release the first actuation element 901 and the second steering element 906 pulls the second actuation element 902 in a proximal direction. The simultaneous pulling and releasing of the actuation elements 901, 902 provides bi-directional movement of a distal end portion of the catheter shaft 912 to which the actuation elements 901, 902 are attached.

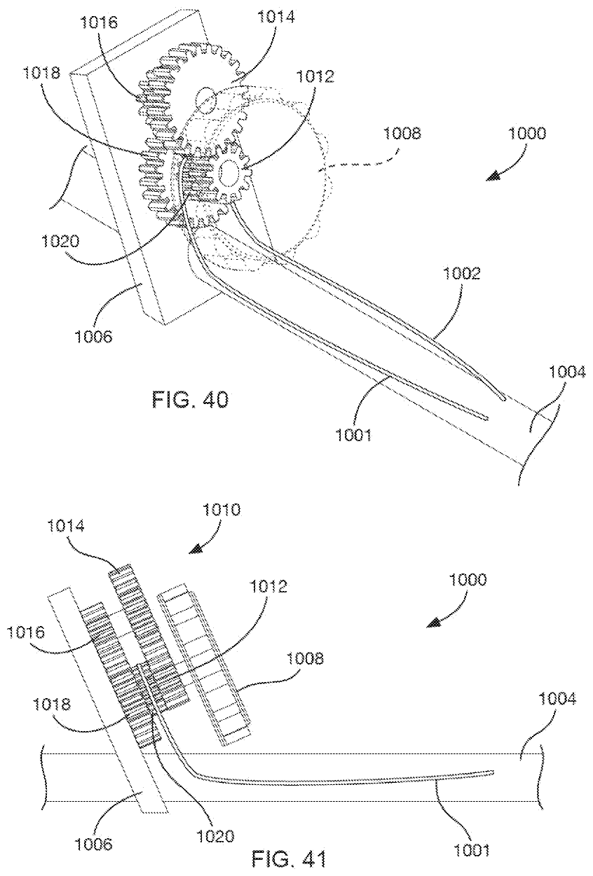

Referring now to FIGS. 40-41, an example steering mechanism 1000 for a delivery system is shown. The steering mechanism 1000 is connected to a proximal end of a catheter shaft 1004 and includes a transmission 1010 for transmitting rotational movement of a control member 1008 to the simultaneously pulling and releasing of actuation elements 1001, 1002 that extend through the catheter shaft 1004. The transmission 1010 is supported by a base 1006 that extends above the catheter shaft 1004 at an angle to provide access to the knob 1008. The steering mechanism 1000 can be housed within a handle (not shown).

The transmission 1010 includes five gears: a first gear 1012, a second gear 1014, a third gear 1016, and a fourth gear 1018, and a fifth gear 1020. The knob 1008, first gear 1012, fourth gear 1018, and fifth gear 1020 are coaxially arranged. The knob 1008 and first gear 1012 have a fixed relationship such that the first gear 1012 rotates when the knob 1008 is rotated. Similarly, the second gear 1014 and third gear 1016 are coaxially arranged have a fixed relationship such that the second gear 1014 and third gear 1016 rotate together. The fourth gear 1018 and fifth gear 1020 also have a fixed relationship and rotate together.

During operation of the steering mechanism 1000, rotation of the knob 1008 at a first speed causes the first gear 1012 to turn at the first speed. Because the second gear 1014 has a larger diameter than the first gear 1012, rotating the first gear 1012 at the first speed causes the second gear 1014 to rotate at a second speed that is slower than the first speed. The third gear 1016 also rotates at the second speed because the third gear 1016 rotates together with the second gear 1014. Rotating the third gear 1016 at the second speed causes the fourth gear 1018 to rotate a third speed that is slower than the second speed because of the larger diameter of the fourth gear 1018 relative to the third gear 1016. The fifth gear 1020 also rotates at the third speed. Thus, the transmission 1010 reduces the rate of rotation from the first speed down to the third speed. This reduction in speed corresponds with a proportional increase in the torque output of the transmission 1010. That is, a smaller torque applied to the knob results in an amplified torque at the fifth gear 1020 by virtue of the mechanical advantage provided by the transmission 1010.

The actuation elements 1001, 1002 can be formed into a single loop that wraps around the fifth gear 1020. The fifth gear 1020 can be formed out of a high friction material, such as rubber, to engage the actuation elements 1001, 1002. An additional toothed belt (not shown) can also be provided that wraps around the fifth gear 1020 to transmit the torque of the knob 1008 through the transmission 1010 and to the actuation elements 1001, 1002. The actuation elements 1001, 1002 can be connected to the toothed belt by any suitable means, such as, with a grasping element disclosed herein. As the actuation elements 1001, 1002 extend from the transmission 1010 to the catheter shaft 1004 the actuation elements 1001, 1002 can be redirected by any suitable means, such as, for example, by eyelets, wheels, tubes, internal bearing surfaces handle, or the like.

When the control member 1008 is rotated in the clockwise direction the fifth gear 1020 similarly rotates in the clockwise direction to pull the first actuation element 1001 in a proximal direction and to release the second actuation element 1002. When the control member 1008 is rotated in the counter-clockwise direction, the fifth gear 1020 rotates in the counter-clockwise direction to release the first actuation element 1001 and to pull the second actuation element 1002 in a proximal direction. The simultaneous pulling and releasing of the actuation elements 1001, 1002 provides bi-directional movement of a distal end portion of the catheter shaft 1004 to which the actuation elements 1001, 1002 are attached.

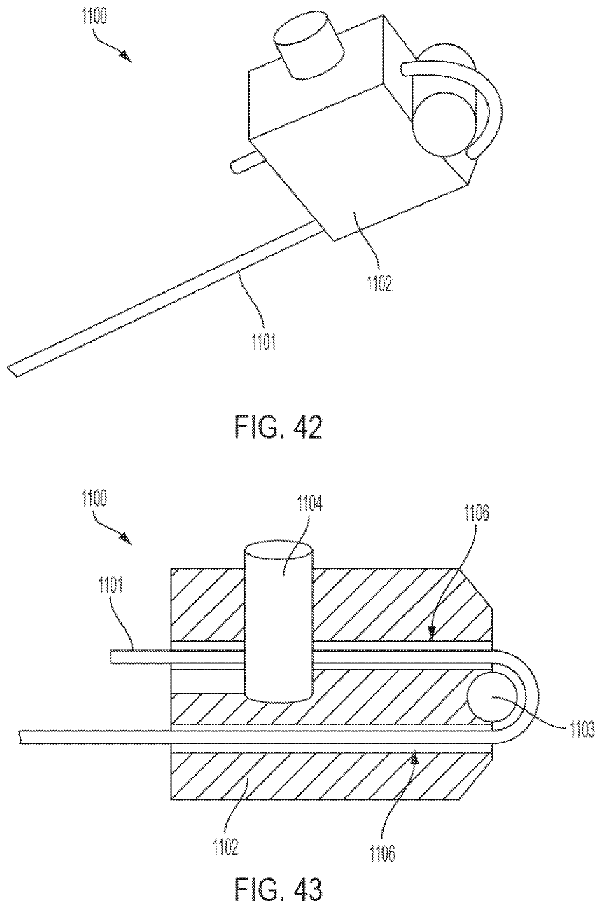

Referring now to FIGS. 42-43, a proximal end of an actuation element 1101 is shown retained by an example grasping element 1102 as part of a delivery system 1100. The actuation element 1101 is a round actuation element 1101. The actuation element 1101 is inserted into one of two slots or openings 1106 of the grasping element 1102, is bent around a bearing member 1103, and is inserted into the other of the two slots or openings 1106 where the end of the actuation element 1101 is retained in position by a set screw 1104. Wrapping the actuation element 1101 around the bearing member 1103 distributes the load across the surface of the bearing member 1103 to reduce the load experienced by the actuation element 1101 at any given point. The bearing member 1103 can be a pin, screw, rivet, or the like.

The grasping element 1102 can be attached to any suitable portion of the delivery system 1100, such as, for example, a clamp or a steering element of a steering mechanism. Optionally, the grasping element 1102 can be integrally formed with any suitable component of the delivery system 1100, such as, for example, the handles and steering mechanisms described herein. The example grasping element 1102 enables the length of the actuation element 1101 to be adjusted by loosening the set screw 1104, repositioning the actuation element 1101, and tightening the set screw 1104 against the actuation element 1101 again. Thus, once the delivery system 1100 is assembled the length and/or tension of the actuation element 1101 can be adjusted.

Referring now to FIGS. 44-47, a proximal end of an actuation element 1201 is shown retained by an example grasping element 1206 as part of a delivery system 1200. The actuation element 1201 is a round actuation element 1201 that is inserted into a slot or opening 1212 of the grasping element 1206 and is retained in position by two set screws 1207. Optionally, the grasping element 1206 can be integrally formed with any suitable component of the delivery system 1200, such as, for example, the handles and steering mechanisms described herein. The grasping element 1206 is retained in a pocket 1205 of a steering element 1209 of a steering mechanism. The pocket 1205 can include an undercut 1211 or other retaining feature such that the grasping element 1206 is held in place and encourages to be retained within the pocket 1205 when the grasping element 1206 is subjected to a tensile load via the actuation element 1201. The actuation element 1201 can be adjusted within the grasping element 1206 by removing the grasping element 1206 from the pocket 1205 of the steering element 1209, loosening the screws 1207, repositioning the actuation element 1201, and retightening the screws 1207. The grasping element 1206 can then be inserted back into the pocket 1205 of the steering element 1209.

Referring now to FIG. 48, an example delivery system 1300 is shown. The delivery system 1300 includes actuation elements 1301, 1302 that extend from steering elements 1304 of a steering mechanism (not shown) to a distal portion 1308 such as, for example, a pull ring, a low-profile pull ring, a hypotube, or the like. The actuation elements 1301, 1302 are attached to the steering elements 1304 by grasping elements 1306 that can take on any suitable form, such as the grasping elements 1306 described herein.

To protect the actuation elements 1301, 1302 and reduce the likelihood of undesirable bending or kinking, a pair of example support structures 1310 surround the actuation elements 1301, 1302 and extend from the steering elements 1304 to the distal portion 1308. The support structures 1310 can be used to support actuation elements of any shape or thickness and of any of the delivery systems disclosed herein over longer unsupported distances to prohibit the actuation elements 1301, 1302 from being damaged from prolapsing, kinking, or the like. The support structures 1310 can be entirely contained within a handle of the delivery system 1300 such that the support structures 1310 do not extend into a catheter shaft. That is, the catheter shaft can serve the purpose of the support structures along the length of the catheter shaft while more space inside the handle may require additional support from the support structures.

The support structures 1310 include a first tube 1320 and a second tube 1330. A distal end 1322 of the first tube 1320 is connected to the distal portion 1308 and a proximal end 1324 of the first tube 1320 overlaps with the second tube 1330. A distal end 1332 of the second tube 1330 overlaps with the first tube 1320 and a proximal end 1334 of the second tube 1330 is connected to the steering elements 1304. Where the first tube 1320 has a smaller diameter than the second tube 1330, the proximal end 1324 of the first tube 1320 extends inside the second tube 1330, or vice versa. Because of the connection of the distal end 1322 of the first tube 1320 to the distal portion 1308 and the connection of the proximal end 1334 of the second tube 1330 to the steering elements 1304 the first and second tubes 1320, 1330 slide relative to each other as the actuation elements 1301, 1302 bend and flex in response to bending and flexing of the catheter or catheter shaft of the delivery system 1300. In other words, the support structures 1310 can be telescoping support structures that can change in length to accommodate changes in the length of the actuation elements 1301, 1302.

The inner diameter of the smaller of the first and second tubes 1320, 1330 can be about 5 percent to about 10 percent greater than the outer diameter of round actuation elements. Where the actuation elements are flat, the inner diameter of the smaller of the first and second tubes 1320, 1330 can be at least the width of the flat actuation elements, such as about 5 percent to about 10 percent greater than the width of the flat actuation elements, or about the same as the width of the flat actuation elements. The support structures 1310 can be formed from any suitable material, such as, for example, a plastic such as PTFE or a metal such as nitinol.

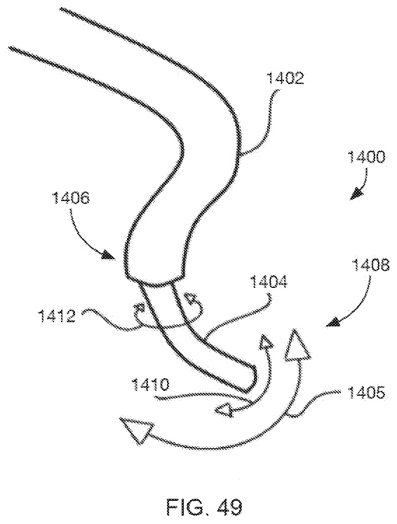

Referring now to FIG. 49, an example steerable catheter 1400 is shown that includes two catheter shafts: an outer shaft 1402 and an inner shaft 1404. The outer shaft 1402 is configured to reach a location above a center of an annulus, e.g., a mitral valve and a tricuspid valve annulus. The outer shaft 1402 has at least two sequentially arranged bending sections (see, e.g., FIGS. 8-13) so that the outer shaft 1402 can be bent and flexed so that a distal end 1406 of the outer shaft 1402 is arranged above and facing the native valve of a patient during an operation. Once the position of the outer shaft 1402 has been fine-tuned, the inner shaft 1404 can be extended, bent, and rotated to place a distal end 1408 of the inner shaft 1404 at a desired location closer to the tissue of the native valve.

The inner shaft 1404 can be controlled to extend distally so that the inner shaft 1404 becomes longer than the outer shaft 1402. Once extended to a desired length, the inner shaft 1404 can be manipulated by a steering mechanism to bend in a lateral direction 1410 and to twist or rotate in an axial direction 1412. These motions can be combined to move the extended and bent inner shaft 1404 in a sweeping motion 1405. Thus, rather than manipulating the distal end 1408 of the inner shaft 1404 via bending in two planes (similar to a cartesian coordinate system) the distal end 1408 of the inner shaft 1404 can be manipulated by bending and rotating (similar to a polar or spherical coordinate system). That is, the position of the distal end 1408 of the inner shaft 1404 can be specified by the extension length, the bend angle, and the rotation or twist angle of the inner shaft 1404. Thus, accessing a different location along the annulus of the native heart valve is a matter of rotating or twisting the inner shaft 1404 in the axial rotation direction 1412 a desired amount while maintaining the same bend angle and extension distance.

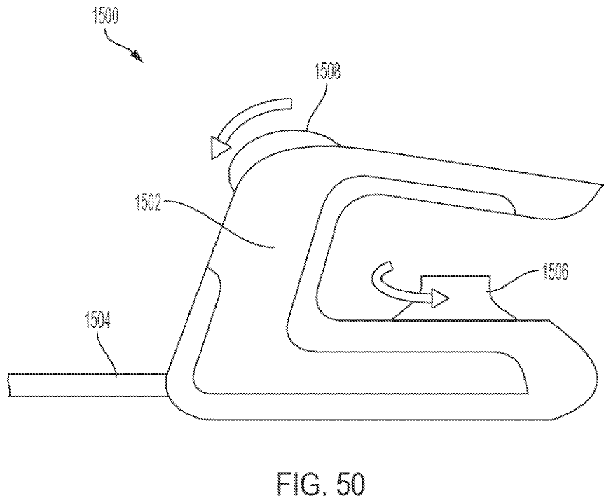

Referring now to FIG. 50, an example control handle 1502 for a delivery system 1500 is shown. The control handle 1502 is attached to a proximal end of a catheter shaft 1504 and includes first and second control members 1506, 1508 for actuating a steering mechanism (not shown) housed by the control handle 1502. The first and second control members 1506, 1508 actuate first and second steering systems that enable the control of first and second sets of actuation elements (not shown) that extend down the catheter shaft 1504 to manipulate a distal end portion of the delivery system 1500, respectively. The control handle 1502 is shaped somewhat like an iron but can take on a wide variety of shapes with varying arrangements of the first and second control members 1506, 1508. Control handles disclosed herein, such as the control handle 1502 of the delivery system 1500 can have a variety of configurations, shapes, and sizes depending on the type of steering mechanism and steering elements located inside the handle and depending on the use of the catheter.

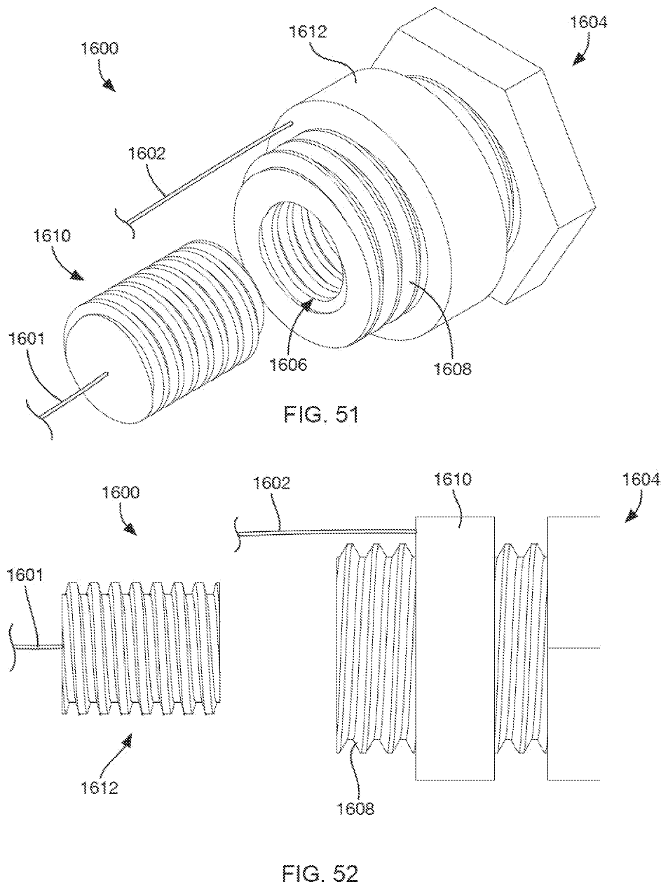

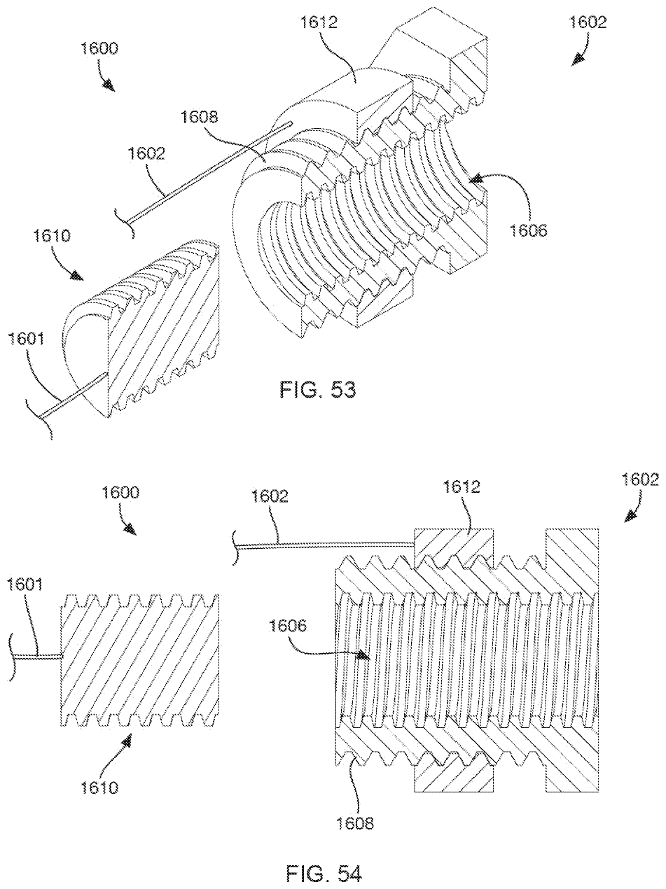

Referring now to FIGS. 51-54, an example steering mechanism 1600 for a delivery system is shown. The steering mechanism 1600 is capable of simultaneously pulling and releasing actuation elements 1601, 1602 and can be used with any delivery system disclosed herein. The steering mechanism 1600 includes a control member 1604 having an internal threaded portion 1606 and an external threaded portion 1608. The threads of the internal and external threaded portions 1606, 1608 are opposite to each other, that is, the internal threaded portion 1606 has a right-hand thread when the external threaded portion 1608 has a left-hand thread, and vice versa. The steering mechanism 1600 further includes a first steering element 1610 having external threads that match those of the internal threaded portion 1606 and a second steering element 1612 having internal threads that match those of the external threaded portion 1608. (The first steering element 1610 threads into the internal threaded portion 1606 of the knob 1604 but is shown disassembled from the knob 1604 for illustration purposes.) The first steering element 1610 is connected to the first actuation element 1601 and the second steering element 1612 is connected to the second actuation element 1602. The actuation elements 1601, 1602 are connected to the steering elements 1610, 1612 by any suitable means, such as welding or with a grasping element disclosed herein.

When the control member 1604 is rotated in the clockwise direction, the first steering element 1610 pulls the first actuation element 1601 in a proximal direction and the second steering element 1612 moves in a distal direction to release the second actuation element 1602. When the control member 1604 is rotated in the counter-clockwise direction, the first steering element 1610 moves in a distal direction to release the first actuation element 1601 and the second steering element 1612 pulls the second actuation element 1602 in a proximal direction. The simultaneous pulling and releasing of the actuation elements 1601, 1602 provides bi-directional movement of a distal end portion of a catheter or catheter shaft to which the actuation elements 1601, 1602 are attached.

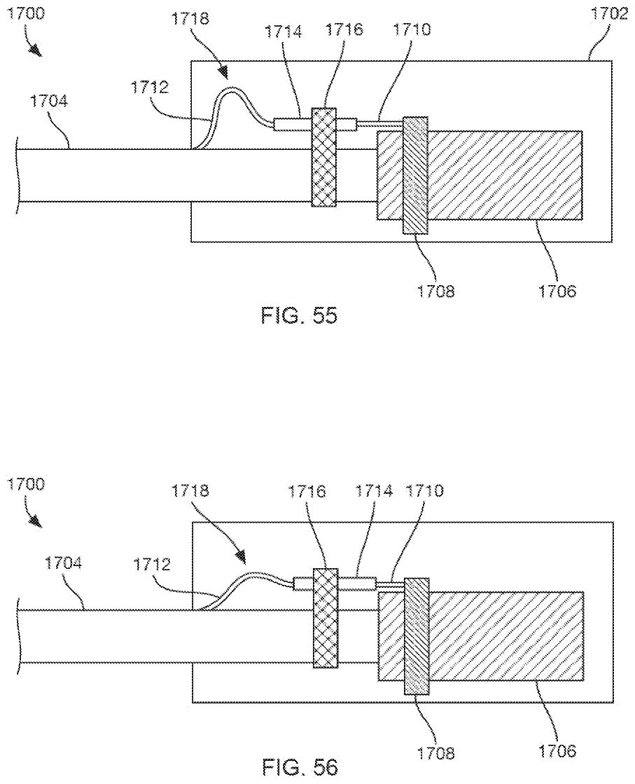

Referring now to FIGS. 55 and 56, an example delivery system 1700 is shown. The delivery system 1700 includes a handle 1702, a catheter shaft 1704 extending from the handle 1702, a steering mechanism 1706 with a steering element 1708 that is connected to an actuation element 1710. The actuation element 1710 extends through a compression member 1712 that extends distally from a stopper 1714, into the catheter shaft 1704, and to a distal end (not shown) of the catheter shaft 1704. In some implementations, the compression member 1712 is a compression coil and the stopper 1714 is a coil stopper. The compression member 1712 can attach to an end of the stopper 1714 or a side of the stopper 1714.

The compression member 1712 transmit compression forces from a distal end of the delivery system 1700 through the catheter shaft 1704 to the handle 1702 to reduce the impact of the compression forces on the performance of the delivery system 1700. The compression forces can be a result of, for example, the retraction of the actuation element 1710 to bend or flex the distal end of the delivery system 1700. The length of the compression member 1712 is tailored to the length of the catheter shaft 1704. A compression member 1712 that is too long can cause friction within the catheter shaft 1704 that makes it difficult to operate the delivery system 1700. A compression member 1712 that is too short can damage the delivery system 1700 and can make it harder to bend or flex the catheter shaft 1704 and otherwise operate the delivery system 1700.

A service loop 1718 formed between the stopper 1714 and the catheter shaft 1704 provides some additional material or slack in the compression member 1712 to accommodate relatively small changes in the length of the compression member 1712 as the catheter shaft 1704 is bent or flexed. The service loop 1718 is formed during initial assembly of the delivery system 1700 wherein the compression member 1712 is installed along the catheter shaft 1704, measured to the appropriate length, marked, and trimmed by the operator. The trimming operation adds time to the procedure and can be a difficult task when the compression member 1712 is formed from a tough or hard material.

A trimming step during assembly of the delivery is not required by the example delivery system 1700 shown in FIGS. 55 and 56. That is, the stopper 1714 at the proximal end of the compression member 1712 of the delivery system 1700 is adjustably connected to the catheter shaft 1704 by a mounting portion 1716 so that the length of the compression member 1712 can be adjusted after the delivery system 1700 is assembled. Consequently, the compression member 1712 can be cut to a desired length during manufacturing so that no trimming of the compression member 1712 is required by the operator. Minor adjustments to the length of the compression member 1712 can then be made by adjusting the position of the stopper 1714. The position of the stopper 1714 relative to the mounting portion 1716 can be adjusted in a wide variety of ways. For example, the stopper 1714 can be formed from threaded body that is threaded into a threaded opening of the mounting portion 1716 so that the stopper 1714 is translated proximally or distally by rotating the stopper 1714. The stopper 1714 can also be retained within an opening of the mounting portion 1716 by set screws, a clamp, or the like.

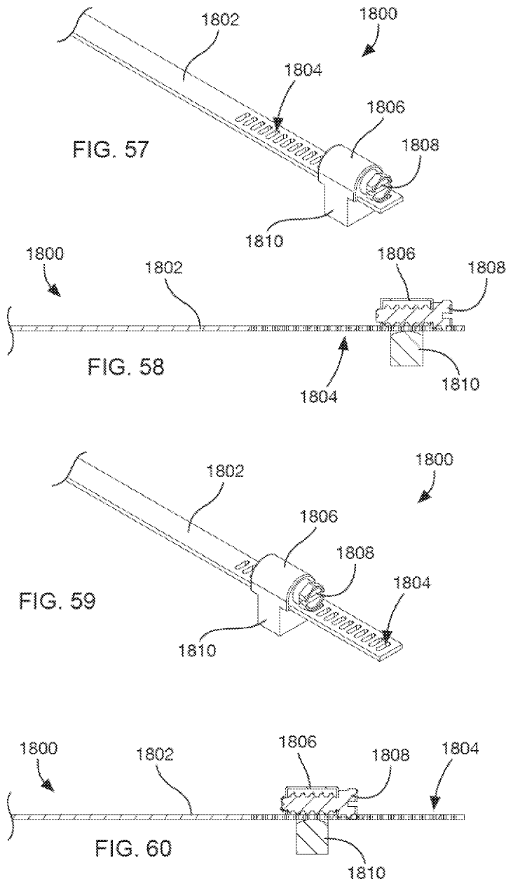

Referring now to FIGS. 57-60, a proximal end of an actuation element 1802 is shown retained by an example grasping element 1806 as part of a delivery system 1800. The actuation element 1802 is a flat actuation element 1802 with a rectangular cross-sectional shape that includes a plurality of evenly spaced apart slots or holes 1804 arranged at the proximal end of the actuation element 1802. The actuation element 1802 is inserted into a slot or opening of the grasping element 1806 such that the spaced apart slots 1804 are engaged by threads of an adjustment screw 1808 retained within the grasping element 1806. The grasping element 1806 further includes a base 1810 that can be attached to any suitable portion of the delivery system 1800, such as, for example, a clamp or a steering element of a steering mechanism. Optionally, the base 1810 can be integrally formed with any suitable component of the delivery system 1800, such as, for example, the handles and steering mechanisms described herein.

The example grasping element 1806 enables the length of the actuation element 1802 to be adjusted via the adjustment screw 1808. Turning the adjustment screw 1808 causes the actuation element 1802 to retract into or extend from the grasping element 1806. Thus, once the delivery system 1800 is assembled the length and/or tension of the actuation element 1802 can be adjusted by tightening or loosening the adjustment screw 1808. In some implementations, the grasping element 1806 can further include a lock washer or cap to prohibit movement of the adjustment screw 1808 after the desired adjustment to the actuation element 1802 has been made.

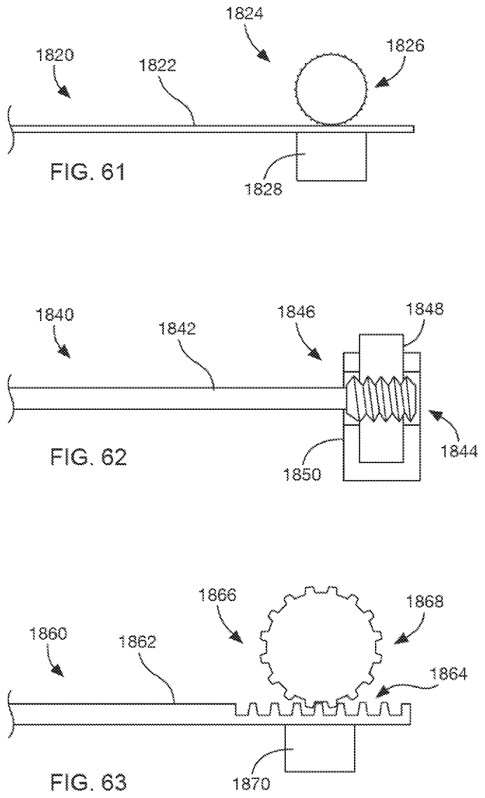

Referring now to FIGS. 61-63, examples of adjustable grasping elements 1824, 1846, 1866 for actuation elements 1822, 1842, 1862 for delivery systems 1820, 1840, 1860, respectively, are shown. Referring now to FIG. 61, the delivery system 1820 includes the actuation element 1822 that is a flat actuation element 1822 that extends through the grasping element 1824. The grasping element 1824 includes a textured wheel 1826 that presses the actuation element 1822 against a base 1828. The textured wheel 1826 engages the surface of the flat actuation element 1822 that may or may not include slots or holes like the actuation element 1802, above. Friction between the textured wheel 1826 and the surface of the actuation element 1822 enables the position of the actuation element 1822 to be adjusted relative to the grasping element 1824, thereby changing the length and/or tension of the actuation element 1822.

Referring now to FIG. 62, the delivery system 1840 includes the actuation element 1842 that is a round actuation element 1842 that includes a threaded end portion 1844 that extends through the grasping element 1846. The threaded end portion 1844 is engaged by a threaded collar 1848 that is rotatably retained by or otherwise connected to a base 1850. Rotating the threaded collar 1848 enables the position of the actuation element 1842 to be adjusted relative to the grasping element 1846, thereby changing the length and/or tension of the actuation element 1842.

Referring now to FIG. 63, the delivery system 1860 includes the actuation element 1862 that is a round actuation element 1862 that includes a plurality of slots 1864, the actuation element 1862 extending through the grasping element 1866. The slots 1864 are formed in one side of the actuation element 1862 or extend around the entire circumference of the actuation element 1862. The slots 1864 are engaged by a toothed wheel or gear 1868 that presses the actuation element 1862 against a base 1870. The engagement of slots 1864 by the toothed wheel 1868 enables the position of the actuation element 1862 to be adjusted relative to the grasping element 1866, thereby changing the length and/or tension of the actuation element 1862.

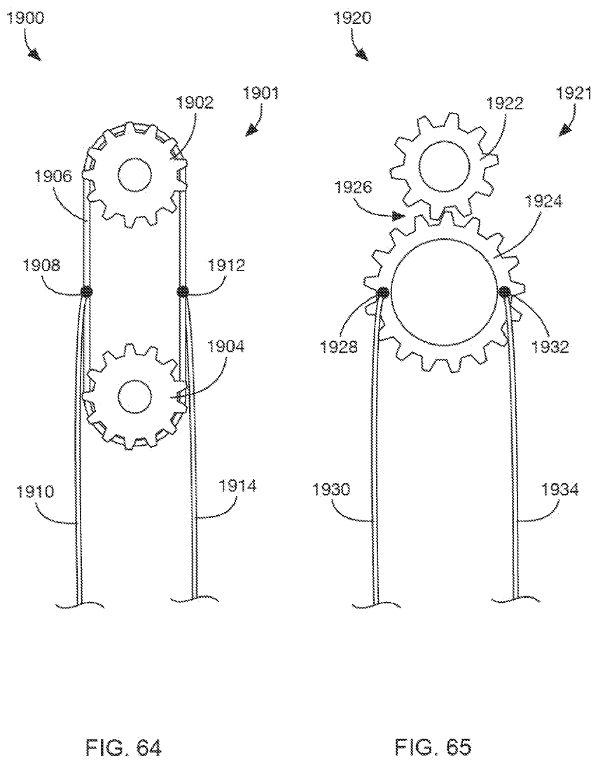

Referring now to FIG. 64, a steering mechanism 1901 for an example delivery system 1900 is shown. The steering mechanism 1901 includes a drive gear 1902, a driven gear 1904, and a drive member 1906. The drive member 1906 can be a chain that engages teeth of the drive and/or driven gears 1902, 1904 or, optionally, a belt that engages the drive and/or driven gears 1902, 1904 that may or may not include teeth. A first attachment member 1908 connects a first actuation element 1910 to one side of the drive member 1906 and a second attachment member connects a second actuation element 1914 to the other side of the drive member 1906. During operation, rotating the drive gear 1902 in a clockwise direction exerts a tension force on the first actuation element 1910 while releasing tension on the second actuation element 1914 and rotating the drive gear 1902 in a counter-clockwise direction exerts a tension force on the second actuation element 1914 while releasing tension on the first actuation element 1910. Thus, the steering mechanism 1901 can be used to actuate a distal end portion of a delivery system to cause the distal end portion to bend or flex. An additional backspin prevention mechanism (not shown) can also be included to prohibit the tension on the actuation elements 1912, 1914 from rotating the drive and/or driven gears 1902, 1904.

Referring now to FIG. 65, a steering mechanism 1921 for an example delivery system 1920 is shown. The steering mechanism 1921 includes a drive gear 1922 and a driven gear 1924 that mesh together at an engagement point 1926. A first attachment member 1928 connects a first actuation element 1930 to one side of the driven gear 1924 and a second attachment member 1932 connects a second actuation element 1934 to the other side of the driven gear 1924. During operation, rotating the drive gear 1922 in a counter-clockwise direction exerts a tension force on the first actuation element 1930 while releasing tension on the second actuation element 1934 and rotating the drive gear 1922 in a clockwise direction exerts a tension force on the second actuation element 1934 while releasing tension on the first actuation element 1930. Thus, the steering mechanism 1921 can be used to actuate a distal end portion of a delivery system to cause the distal end portion to bend or flex. An additional backspin prevention mechanism (not shown) can also be included to prohibit the tension on the actuation elements 1932, 1934 from rotating the drive and/or driven gears 1922, 1924.

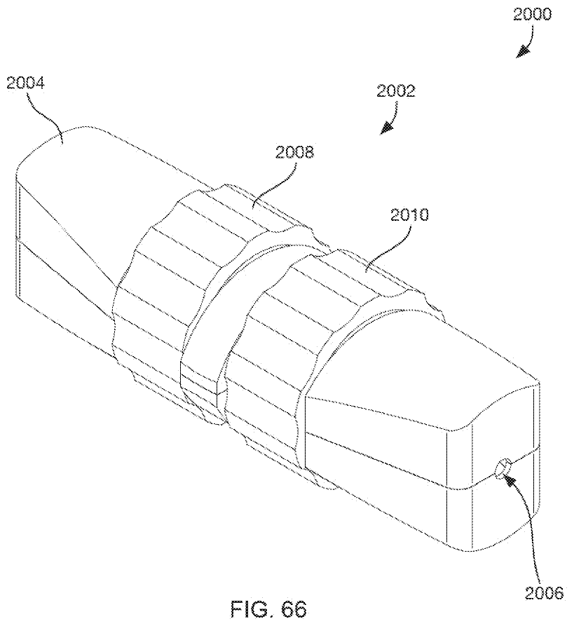

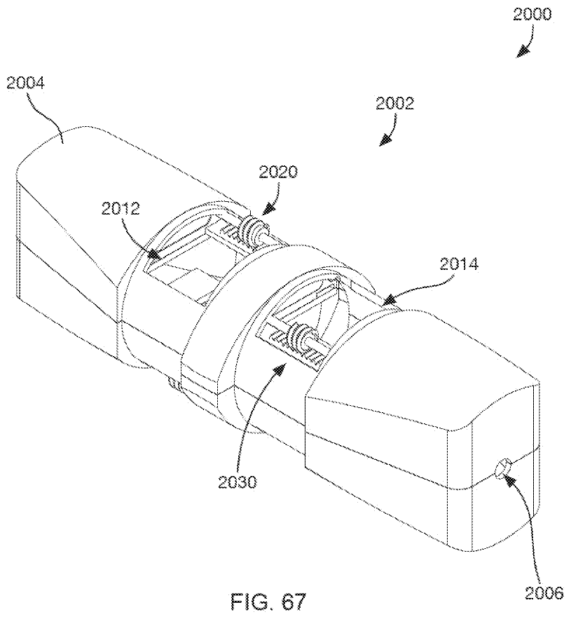

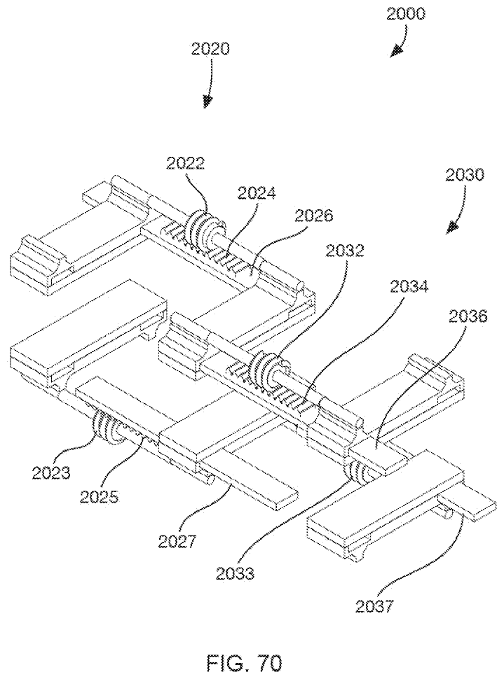

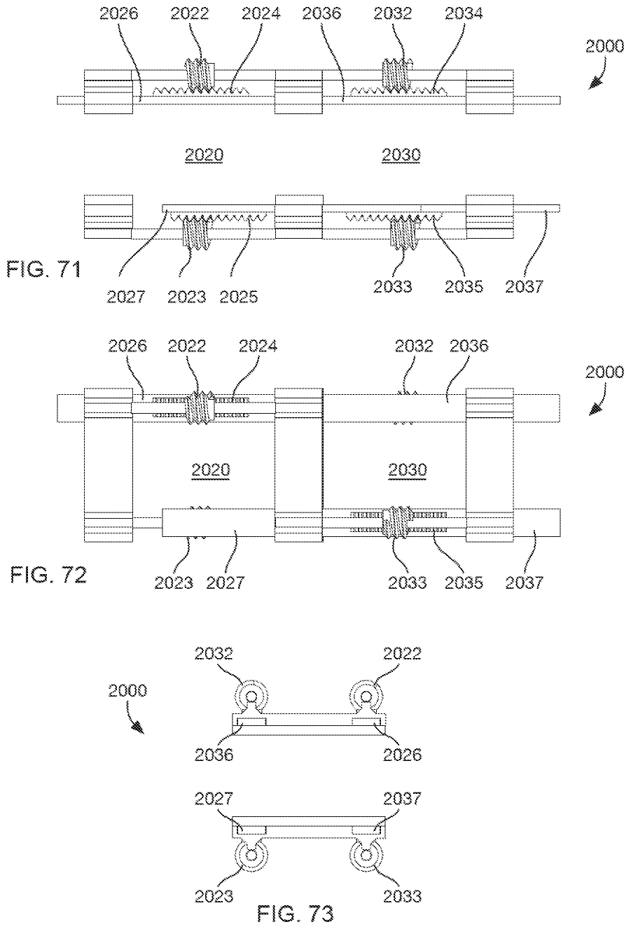

Referring now to FIGS. 66-73, a control handle 2002 including steering mechanisms 2020, 2030 (FIGS. 67-73) for an attached catheter or catheter shaft (not shown in FIGS. 66-73) of an example delivery system 2000 are shown. The control handle 2002 can be attached to a catheter, catheter shaft, or other elongated and steerable tubular or transluminal device for insertion into a patient. The control handle 2002 is an omni-directional control handle in that the control handle 2002 facilitates control of a distal end portion of a catheter shaft to be bent or flexed in any direction, that is, in a full 360-degree circle around the catheter shaft.

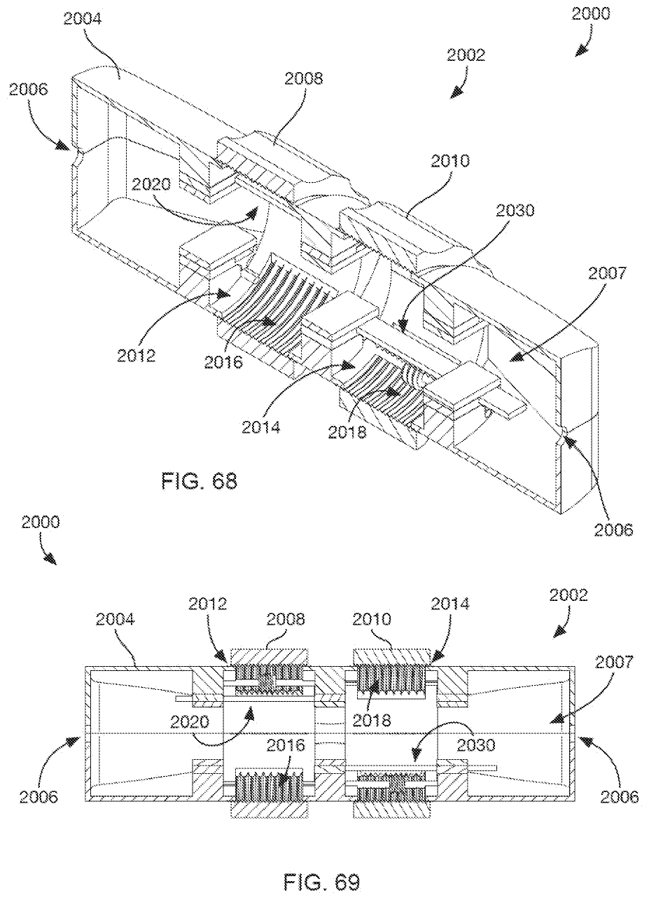

The control handle 2002 includes a housing 2004 that has openings 2006 on each end through which actuation elements (not shown) extend from the control handle 2002 to a catheter shaft (not shown). The openings 2006 form a luminal access 2007 (FIGS. 68 and 69) that extends longitudinally through the control handle 2002 for passage of other devices, actuation elements, and/or fluids through the handle 900 and the attached catheter.

The handle includes one or more control members, e.g., a first control member 2008 (e.g., a knob, button, switch, gear, etc.) and a second control member 2010 (e.g., a knob, button, switch, gear, etc.).

In some implementations, the first control member 2008 and the second control member 2010 are arranged concentrically with a longitudinal axis of the control handle 2002 and are rotatably attached to the housing 2004. The arrangement of the first and second control members 2008, 2010 on the exterior of the control handle 2002 provides the operator with increased leverage when actuating the control members 2008, 2010. That is, the diameter of the control members 2008, 2010 is greater and therefore provides more mechanical advantages than knobs that are disposed within a control handle.

Actuating (e.g., rotating, etc.) the first and second control members 2008, 2010 actuates first and second steering mechanisms 2020, 2030, respectively, that are contained inside the control handle 2002. The first and second steering mechanisms 2020, 2030 can be the same as or similar to other steering mechanisms described herein.

In some implementations, the first control member 2008 covers first actuation openings 2012 that provide access to the first steering mechanism 2020. An interior thread 2016 of the first control member 2008 engages first and second drive gears 2022, 2023 of the first steering mechanism 2020 to cause the drive gears 2022, 2023 to rotate. When rotated, the drive gears 2022, 2023 engage first and second racks 2024, 2025 that are connected to first and second steering elements 2026, 2027 to cause the first and second steering elements 2026, 2027 to extend or retract. The first and second drive gears 2022, 2023 are threaded in opposite directions and engage opposite threaded portions of the interior thread 2016 of the knob 2008 so that rotation of the first control member 2008 causes the first drive gear 2022 to rotate opposite the second drive gear 2023. Consequently, the first and second steering elements 2026, 2027 move in opposite axial directions when the first control member 2008 is rotated. That is, the first steering element 2026 extends as the second steering element 2027 retracts, and vice versa. Actuation elements attached to the steering elements 2026, 2027 that move proximally with respect to the housing 2004 increase in tension and actuation elements attached to steering elements 2026, 2027 that move distally relax. The attached catheter or catheter shaft flexes in the direction of the tensed actuation elements. Thus, control of magnitude and direction of flex is not independent for control handle 2002.

The second control member 2010 covers second actuation openings 2014 that provide access to the second steering mechanism 2030. An interior thread 2018 of the second control member 2010 engages first and second drive gears 2032, 2033 of the second steering mechanism 2030 to cause the drive gears 2032, 2033 to rotate. When rotated, the drive gears 2032, 2033 engage first and second racks 2034, 2035 that are connected to first and second steering elements 2036, 2037 to cause the first and second steering elements 2036, 2037 to extend or retract. The first and second drive gears 2032, 2033 are threaded in opposite directions and engage opposite threaded portions of the interior thread 2018 of the knob 2010 so that rotation of the second control member 2010 causes the first drive gear 2032 to rotate opposite the second drive gear 2033. Consequently, the first and second steering elements 2036, 2037 move in opposite axial directions when the second control member 2010 is rotated. That is, the first steering element 2036 extends as the second steering element 2037 retracts, and vice versa. Actuation elements attached to the steering elements 2036, 2037 that move proximally with respect to the housing 2004 increase in tension and actuation elements attached to steering elements 2036, 2037 that move distally relax. The attached catheter or catheter shaft flexes in the direction of the tensed actuation elements. Thus, control of magnitude and direction of flex is not independent for control handle 2002.

The steering elements 2026, 2027, 2036, 2037 of the first and second steering mechanisms 2020, 2030 can be connected to the proximal ends of actuation elements via grasping elements, such as those disclosed herein, or can be portions of the actuation elements themselves. For example, the actuation elements can be flat actuation elements that are welded or otherwise attached to the racks 2024, 2025, 2034, 2035 of the first and second steering mechanisms 2020, 2030. While the racks 2024, 2025, 2034, 2035 are shown as toothed portions, the racks 2024, 2025, 2034, 2035 could also be formed from a plurality of slots that are engaged by the threads of the drive gears 2022, 2023, 2032, 2033 in the same fashion as the adjustable grasping element 1806 described above and shown in FIGS. 57-60.

The steering elements 2026, 2027, 2036, 2037 are arranged in opposing pairs: first and second steering elements 2026, 2027 of the first steering mechanism 2020; and first and second steering elements 2036, 2037 of the second steering mechanism 2030. The opposing pairs of steering elements 2026, 2027, 2036, 2037 are arranged 180 degrees from each other so that the first steering elements 2026, 2036 are opposite the second steering elements 2027, 2037. Thus, the first knob 2008 controls flexing of an attached catheter or catheter shaft in a first bending plane and the second knob 2010 controls flexing of an attached catheter or catheter shaft in a second bending plane that is orthogonal to the first bending plane. Combining bend magnitudes in each of the first and second bending planes enables the catheter or catheter shaft to be bent or flexed in any direction.

While the steering mechanisms 2020, 2030 are shown with manually actuated knobs 2008, 2010, the steering mechanisms 2020, 2030 can be actuated by other means, such as, for example, electrical motors or other actuators. Additionally, it should be noted that additional combinations of steering mechanisms and knobs can be stacked longitudinally with the first and second steering mechanisms to provide controls for additional mechanisms and/or to control bending in additional bending planes.

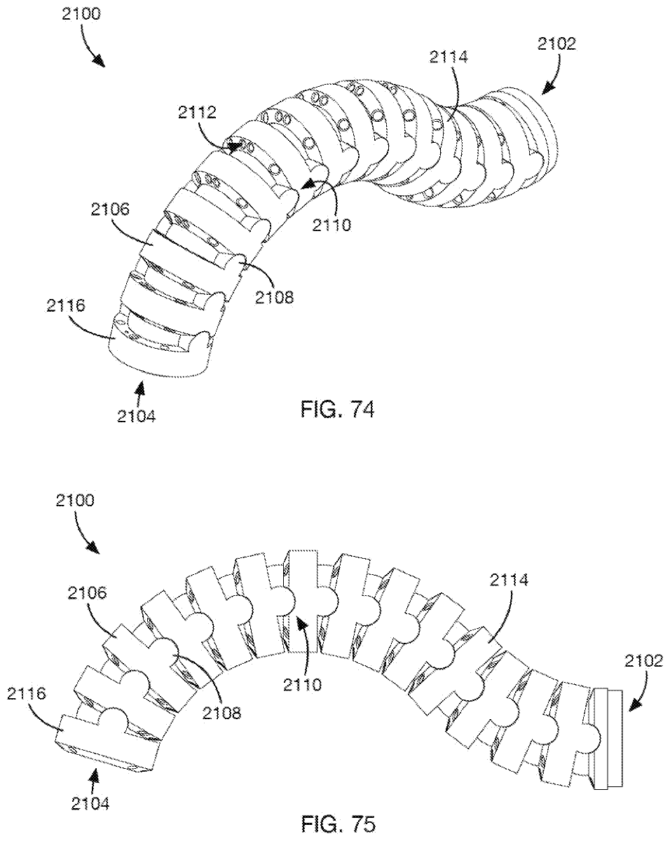

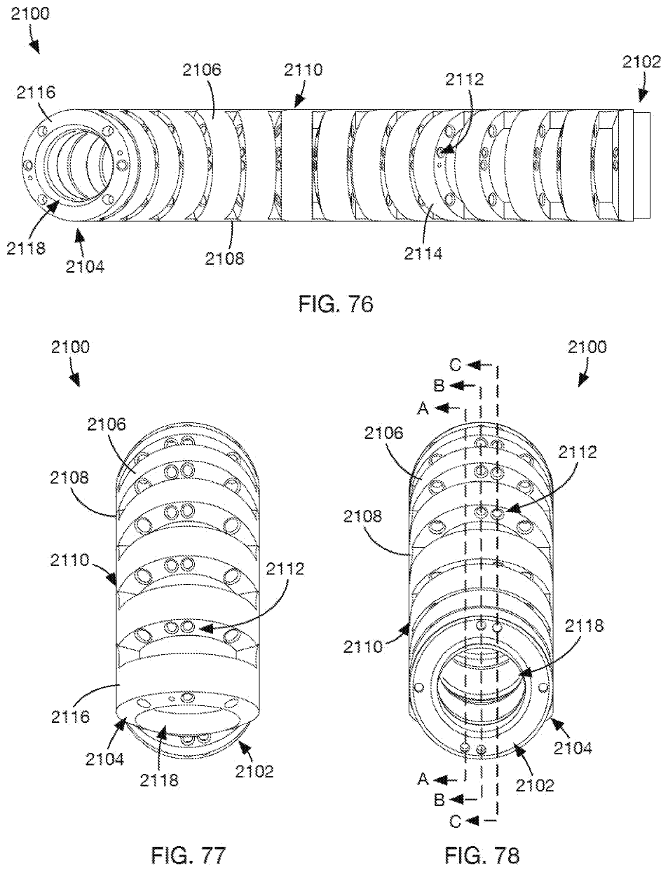

Referring now to FIGS. 74-82, a portion of an example delivery system 2100 or a portion of the catheter shaft of the delivery system 2100 is shown. The delivery system 2100 or catheter shaft includes a distal end portion that extends from a proximal region or proximal end 2102 of the distal end portion to a distal region or distal end 2104 of the distal end portion. The distal end portion comprises a plurality of links 2106 arranged at a distal end of a catheter shaft of the delivery system 2100.

In some implementations, the links 2106 are configured to operate similar to vertebrae of the human spine. In some implementations, the links 2106 interconnect and can articulate relative to each other. In some implementations, the links 2106 include hinges or joints connecting the links. In some implementations, the links 2106 include protrusions 2018 (shown as convex male protrusions, though other shapes and sizes are possible) that fit together with sockets or recesses 2110 (e.g., shown as concave female recesses, though other shapes and sizes are possible), to provide a pivoting joint between adjacent links 2106.

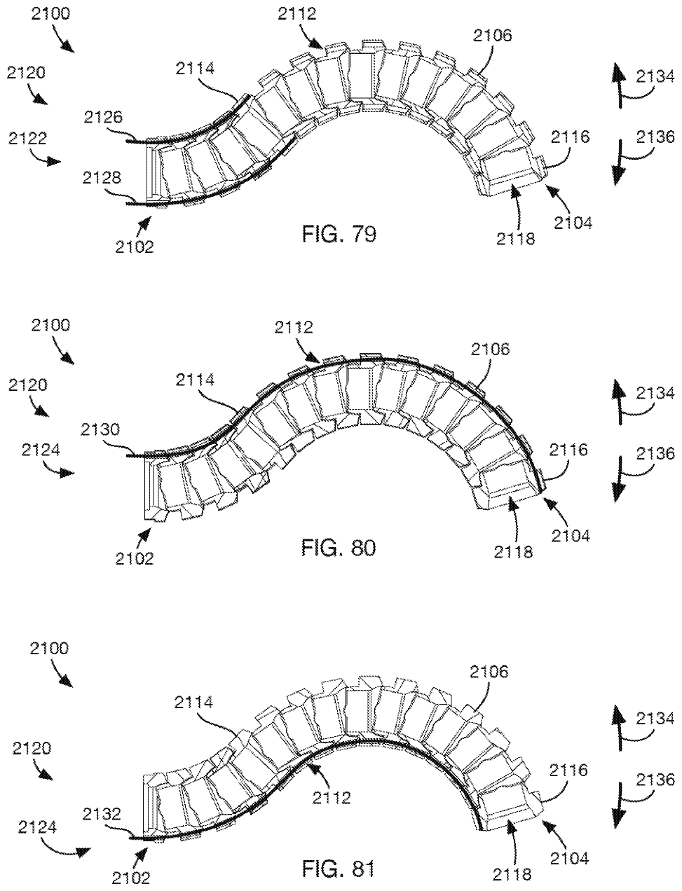

In some implementations, each link 2106 includes a central opening or lumen 2118 and openings or lumen 2112 for guiding and supporting actuation elements 2120 (FIGS. 79-81) that articulate the delivery device 2100.

In some implementations, an optional hypotube (not shown) can also be provided that extends through the central openings 2118 of the links 2106.

In some implementations, one or more sleeves, polymers, laminates, coatings, liners, etc. are located outside and/or inside the links 2106. In some implementations, sleeves, polymers, coatings, liners, etc. sandwich the links 2106 between them.

In some implementation, protrusions 2108 and sockets or recesses 2110 (or other joint or hinge portions) prohibit relative rotation of the links 2106 except along the longitudinal axis of the delivery system 2100 (i.e., along the line B-B of FIG. 78).

In some implementations, actuation elements 2120 can be actuated to bend one or more portions or all of the delivery system 2100 in a first direction 2134 and/or a second direction 2136. For example, applying tension to one or more actuation elements 2120 can cause a link 2106 to which an actuation element 2120 is attached to move or tilt in the direction of the net tension force applied to the actuation element 2120. Consequently, the plurality of links 2106 are caused to pivot relative to each other so that the device 2100 flexes or bends toward the applied force.

In some implementations, the relative proportion of tension applied to two opposingly arranged actuation elements 2120—independent of the amount of tension applied—determines bend or curve direction. The amount of tension difference between the actuation elements 2120, however, is directly related to the magnitude of the bend or curve in the device 2100; the greater the tension imbalance the greater the bend or curve magnitude (i.e., the tighter or smaller the radius of curvature of the bend or curve).

The length of the delivery system 2100 bent or curved by the actuation elements 2120 depends on the link 2106 to which the actuation element(s) 2120 is connected. That is, applying tension to one of the actuation elements 2120 applies bending or curving forces to the links 2106 arranged proximate to the connected link 2106. Thus, the end of the catheter or catheter shaft of the delivery system 2100 can be articulated into a wide variety of positions. The actuation elements can be connected to any one or more of the links to generate different curving or bending configurations.

Referring now to FIG. 79, in some implementations, a first actuation element 2126 and/or a first pair of actuation elements 2122 extends through the proximal end 2102 of the delivery system 2100 to a transition link or intermediate link 2114.

In some implementations, a first pair of actuation elements 2122 includes a first actuation element 2126 and a second actuation element 2128 that are connected to opposite sides of the transition or intermediate link 2114. The first actuation element 2126 and the second actuation element 2128 extend through lumen 2112 arranged along a central plane of the delivery system 2100, as can be seen in FIG. 78. Tension applied to the first actuation element 2126 and/or to the second actuation element 2128 actuates or bends the delivery system 2100 between the proximal end 2102 and the transition or intermediate link 2114 in the direction of the tension force applied to the first actuation element 2126 and/or the second actuation element 2128. That is, if more tension is applied to the first actuation element 2126, then the transition or intermediate link 2114 bends or curves in the first direction 2134, and if more tension is applied to the second actuation element 2128, the transition or intermediate link bends or curves in the second direction 2136. In some implementations, a steering mechanism (which can be the same as or similar to other steering mechanisms herein) can be actuated (e.g., with a control member, etc.) to tension the first actuation element 2126 while releasing tension in the second actuation element 2128, and/or be actuated to tension the second actuation element 2128 while releasing tension in the first actuation element 2126. In some implementations, only a first actuation element 2126 connects to the transition or intermediate link 2114 such that when tension is applied bending or curving at link 2114 occurs in only the first direction 2134.

Referring now to FIGS. 80 and 81, in some implementations a second actuation element and/or a second pair of actuation elements 2124 extends through the proximal end 2102 of the delivery system 2100 to a distal link 2116 at the distal end 2104 of the delivery system 2100.

In some implementations, a second pair of actuation elements 2124 includes a third actuation element 2130 and a fourth actuation element 2132 that are connected to opposite sides of the distal link 2116. The third actuation element 2130 and the fourth actuation element 2132 extend through lumen 2112 arranged adjacent to a central plane of the delivery system 2100, as can be seen in FIG. 78. Tension applied to the third actuation element 2130 and/or to the fourth actuation element 2132 actuates or bends the delivery system 2100 between the proximal end 2102 and the distal link 2116 in the direction of the tension force applied to the third actuation element 2130 and/or the fourth actuation element 2132. That is, if more tension is applied to the third actuation element 2130, the distal link 2116 bends or curves in the first direction 2134, and if more tension is applied to the fourth actuation element 2132, the distal link 2116 bends or curves in the second direction 2136. In some implementations, a steering mechanism (which can be the same as or similar to other steering mechanisms herein) can be actuated (e.g., with a control member, etc.) to tension the third actuation element 2130 while releasing tension in the fourth actuation element 2132, and/or be actuated to tension the fourth actuation element 2132 while releasing tension in the third actuation element 2130. In some implementations, only actuation element 2132 connects to the distal link 2116 such that when tension is applied bending or curving at link 2116 occurs in only the second direction 2136.

In some implementations, the second pair of actuation elements 2124 can be actuated to bend the entire length of the delivery system 2100 to move the distal end 2116 in the first direction 2134 or the second direction 2136. When only the second pair of actuation elements 2124 is actuated to bend the delivery system 2100, the distal end 2104 is displaced laterally away from the plane of the delivery system 2100 extending from the proximal end 2102. As can be seen in FIGS. 79-81, actuating one of the actuation elements 2120 of the first pair of actuation elements 2122 (i.e., the first actuation element 2126) in an opposite direction from the actuation element 2120 of the second pair of actuation elements 2124 (i.e., the fourth actuation element 2132) maintains the distal end 2104 of the delivery system 2100 in nearly the same plane as the proximal end 2102 of the delivery system 2100.

In some implementations, pairs of actuation elements are not connected to a single link. For example, in some implementations, only a first actuation element 2126 connects to the transition link 2114 such that when tension is applied bending or curving at link 2114 occurs in only the first direction 2134, and only a second actuation element 2132 connects to the distal link 2116 such that when tension is applied bending or curving at link 2116 occurs in only the second direction 2136. Much of what is described herein about the function of pairs of actuation elements can also be accomplished with individual actuation elements at link 2114 and link 2116.

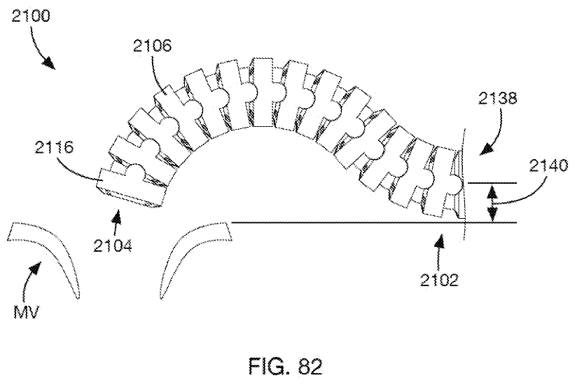

Referring now to FIG. 82, a schematic side view of the distal end portion of delivery system 2100 is shown protruding through a septal puncture or opening 2138 in the septum of the heart and bending toward the mitral valve MV. The trans-septal delivery technique is one technique that can be used to deliver implantable prosthetic devices within the mitral valve. During the trans-septal technique, the delivery device 2100 is extended through the inferior vena cava IVC (see FIGS. 1 and 2) and then through the septal puncture or opening 2138. The height of the distal end of the device 2100 when bent to a maximum bending condition (e.g., 90 degrees) determines a minimum distance between the mitral valve and the puncture through the septum that is made during implantation, i.e., a septal puncture height 2140. If the puncture through the septum is made too close to the mitral valve—below the minimum septal puncture height—the distal end may not be able to bend properly or angle properly for delivery of the medical device or for the desired treatment. Some treatments require the catheter or catheter shaft to be a certain height above or angle relative to the native valve that may not be possible if the catheter can only bend or curve toward the valve from a low septal crossing. For example, some procedures require the catheter or catheter shaft to bend or curve 90 degrees, but a low septal crossing may make it impossible for the catheter to bend or curve to 90 degrees without contacting the tissue of the heart, thereby frustrating proper alignment and implantation of the implantable prosthetic device in the mitral valve. The ability to bend or curve the delivery system 2100 at two locations in or along the same plane enables delivery of an implantable device during a trans-septal delivery, even when the septal puncture or opening 2138 is made at or below the minimum desired septal puncture height 2140. A first bend or curve in a first direction 2134 and a second bend along the same plane in a second direction 2136 allows the catheter shaft of the delivery system to flex away from the native valve before flexing back toward the native valve for a better approach, angle, and/or control at the native valve.

The actuation elements 2120 can be connected at a proximal end to one or more steering elements of a steering mechanism, such as any of the example steering mechanisms disclosed herein, that is arranged in a handle (not shown) at a proximal end of the catheter or catheter shaft. For example, a first steering mechanism can be used to control the bending of the device 2100 between the proximal end 2102 and the transition link 2114 and a second steering mechanism can be used to control the bending of the device 2100 between the transition link 2114 and the distal link 2116.

In an example implementation, a first steering mechanism is connected to the first actuation element 2126 and the second actuation element 2128 that are spaced apart by 180 degrees around the device 2100 and a second steering mechanism is connected to the third actuation element 2130 and the fourth actuation element 2132 that are also spaced apart by 180 degrees around the device 2100.

While two different steering mechanisms are used in various implementations, both steering mechanisms control bending of the device 2100 in or along the same plane because of the protrusions 2108 and recesses 2110, as described above. Optionally, a single steering mechanism can control bending of the device up to the transition link 2114 and between the transition link 2114 and the distal link 2116. The steering mechanism or mechanisms can be arranged in a single handle or in multiple handles such that each handle contains a single steering mechanism.

The actuation elements 2120 can extend through compression members or coils (not shown) that extend from a handle or proximate the handle to a proximal portion of the most proximal link 2106. These can be the same as or similar to other arrangements described or shown herein. The proximal side of the link 2106 can include pockets or recesses for receiving a distal end of the compression member. Optionally, the compression member can run the entire length of the catheter shaft. In any of the catheter implementations herein, each compression member can run through an individual lumen in a shaft of the catheter so that flexing of the shaft does not hinder independent movement of the compression member. In some implementations, the proximal face of a hypotube and/or links of a hypotube have bores and/or extensions to accept or abut against the compression members. The proximal face of the most proximal link 2106 can also have bores and/or extensions to accept or abut against the compression members.

The device 2100 can further include stiffening members (not shown) arranged between the links 2106. The stiffening members cause the device 2100 to be biased in an extension direction so that the links 2106 tend to straighten out after tension applied to the actuation elements 2120 is relieved. The stiffening members can be formed in a tube shape from a shape-memory alloy, such as nitinol. Like the springs 314 of the device 300 shown in FIG. 18, the stiffening members can be springs that are biased in an expanding direction so that as the device 2100 tends to straighten as tension applied to the actuation elements 2120 is relieved. The springs can be arranged between each pair of adjacent links 2106 or can extend through multiple links 2106. Four springs can be arranged between each pair of adjacent links 2106 so that the springs are radially spaced apart by about 90 degrees. Evenly spacing the springs around the circumference of the links 2106 evens out the forces applied to the links 2106 and helps to maintain a symmetrical distance between adjacent rings 2106.

While various inventive aspects, concepts and features of the disclosures may be described and illustrated herein as embodied in combination in the example embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the embodiments in the specification.

What is claimed is:

1. A delivery system for delivering a medical device to a desired location, the delivery system comprising:
a handle;
a catheter shaft extending from a proximal end attached to the handle to a flexible distal end portion;
a steering mechanism attached to the handle for steering the flexible distal end portion of the catheter shaft, the steering mechanism comprising:
a steering element;
a control member;
a first actuation element extending from a proximal end attached to the steering element to a distal end attached to the flexible distal end portion of the catheter shaft;
a second actuation element extending from a proximal end attached to the steering element to a distal end attached to the flexible distal end portion of the catheter shaft;
wherein actuating the control member of the steering mechanism moves the steering element to increase tension in one of the first and second actuation elements and to release tension in the other of the first and second actuation elements; and
wherein actuation of the control member facilitates bi-directional movement of the flexible distal end portion of the catheter shaft.

2. The delivery system of claim 1, wherein the flexible distal end portion comprises a plurality of links, rings, or vertebrae, and wherein the first actuation element attaches to an intermediate ring, link, or vertebrae of the plurality of links, rings, or vertebrae and the second actuation element attaches to a distal ring, link, or vertebrae at or near a distal end of the plurality of links, rings, or vertebrae.

3. The delivery system of claim 2, wherein applying tension to the first actuation element bends the catheter shaft in a first direction and applying tension to the second actuation element bends the catheter shaft in a second direction, wherein the first direction and the second direction are in or along the same plane.

4. The delivery system of claim 2, wherein the plurality of links, rings, or vertebrae include protrusions and recesses configured to connect such that they inhibit rotation of the plurality of links, rings, or vertebrae except along a longitudinal axis of the delivery system.

5. The delivery system of claim 3, wherein the first direction and the second direction are opposite directions.

6. The delivery system of claim 2, wherein in a fully bent condition, the distal end of the plurality of rings or vertebrae is arranged in or along the same plane as a proximal end of the plurality of rings or vertebrae.

7. The delivery system of claim 1, wherein actuation of the control member moves the steering element so that tension is applied to one of the first and second actuation elements and is relieved from the other of the first and second actuation elements.

8. The delivery system of claim 1, wherein actuation of the control member moves the steering element so that tension is applied to one of the first and second actuation elements and compression is applied to the other of the first and second actuation elements.

9. The delivery system of claim 1, wherein the control member engages the steering element via a gear mechanism.

10. The delivery system of claim 1, wherein the first actuation element and the second actuation element are formed from a single pull wire.

11. A delivery system for delivering a medical device to a desired location, the delivery system comprising:
a handle;
a catheter shaft extending from a proximal end attached to the handle to a flexible distal end portion;
a steering mechanism attached to the handle for steering the flexible distal end portion of the catheter shaft, the steering mechanism comprising:
a steering element;
a control member;
a first actuation element extending from a proximal end attached to the steering element to a distal end attached to the flexible distal end portion of the catheter shaft;
a second actuation element extending from a proximal end attached to the steering element to a distal end attached to the flexible distal end portion of the catheter shaft;
wherein actuating the control member of the steering mechanism moves the steering element to increase tension in one of the first and second actuation elements and to release tension in the other of the first and second actuation elements;
wherein the steering mechanism comprises:
a drive gear; and
a driven gear.

12. The delivery system of claim 11, further comprising:
a drive member extending between and engaging the drive gear and the driven gear; and
wherein the first and second actuation elements are attached to the drive member.

13. The delivery system of claim 11, wherein the first and second actuation elements are attached to the driven gear, and wherein the driven gear comprises:
a first rack attached to the first actuation element; and
a second rack attached to the second actuation element.

14. A delivery system for delivering a medical device to a desired location, the delivery system comprising:
a handle;
a catheter shaft extending from a proximal end attached to the handle to a flexible distal end portion;
a steering mechanism attached to the handle for steering the flexible distal end portion of the catheter shaft, the steering mechanism comprising:
a steering element;
a control member;
a first actuation element extending from a proximal end attached to the steering element to a distal end attached to the flexible distal end portion of the catheter shaft;
a second actuation element extending from a proximal end attached to the steering element to a distal end attached to the flexible distal end portion of the catheter shaft;
wherein actuating the control member of the steering mechanism moves the steering element to increase tension in one of the first and second actuation elements and to release tension in the other of the first and second actuation elements; and
wherein the flexible distal end portion comprises a first bending portion and a second bending portion.

15. The delivery system of claim 14, wherein bending characteristics of the first bending portion are different than bending characteristics of the second bending portion.

16. The delivery system of claim 15, wherein the first bending portion has a stiffness that is different from a stiffness of the second bending portion.

17. The delivery system of claim 14, wherein the first bending portion has a length that is different from a length of the second bending portion.

18. The delivery system of claim 14, wherein the first bending portion has a wall thickness that is different from a wall thickness of the second bending portion.

19. The delivery system of claim 14, wherein the flexible distal end portion comprises:
a hypotube extending from the catheter shaft to a distal end;
a plurality of lateral cuts in the hypotube; and
an anchor portion at the distal end of the hypotube for attaching the first actuation element and the second actuation element of the steering mechanism.

20. The delivery system of claim 19, wherein a plurality of cuts in the first bending portion have a spacing that is different than a plurality of cuts in the second bending portion.
